(19) United States
(12) Patent Application Publication
(10) Pub. No.: US 2007/0112273 A1
(43) Pub. Date: May 17, 2007

(54) Measurement system and method for use in determining the patient's condition

(75) Inventor: Steven R. ROGERS, Moshav Beit Gamliel (IL)

Correspondence Address: BROWDY AND NEIMARK, P.L.L.C.;624 NINTH STREET, NW, SUITE 300, WASHINGTON, DC, 20001-5303, US

(73) Assignee: Chameleon Medical Innovation, Ltd., Lod (IL)

(21) Appl. No.: 10/579,631

(22) PCT Filed:
(86) PCT No.: PCT/IL04/01064

Related U.S. Application Data
(60) Provisional application No. 60/520,672, filed on Nov. 18, 2003.

(51) Int. Cl. A61B 6/00; A61B 5/00

(52) U.S. Cl. 600/475; 600/559

(57) ABSTRACT

A measurement system and method are presented for use in detecting a predetermined condition of a patient's ear indicative of a certain disease such as SOM and SOM. The system comprises an optical measuring unit and a control unit connectable to the output of the measuring unit. The optical measuring unit is configured and operable for irradiating a region of interest in a patient's ear with incident light including at least two different wavelengths, detecting light responses of the region of interest to said at least two different wavelengths, and producing measured data indicative thereof. The at least two different wavelengths are selected such that the light response of the region of interest to at least one first wavelength is substantially independent of said predetermined condition and the light response to at least one second wavelength is affected by said predetermined condition. The control unit is operable for receiving and processing the measured data to generate output data indicative of the measurement results. The control unit is configured and operable for controlling operation of the optical measuring unit, and for receiving the measured data and processing it to generate output data indicative of whether or not said predetermined condition exists. The control unit comprises a memory utility for storing predetermined reference data indicative of the light response of the region of interest while in a healthy condition of a patient's ear; a data processing and analyzing utility preprogrammed for processing and analyzing the measured data by determining a relation between the measured light responses and the corresponding reference data.

### FIELD OF THE INVENTION

This invention relates to an optical measurement system and method, as well as an optical probe to be utilized in such system, for use in determining the patient's condition, particularly the ear condition.

### BACKGROUND OF THE INVENTION

Non-invasive optical measurements on a patient's body have been developed and are disclosed for example in the following patent publications: U.S. Pat. No. 4,882,492; U.S. Pat. No. 5,001,556; U.S. Pat. No. 5,280,788; U.S. Pat. No. 5,379,764; U.S. Pat. No. 5,582,168; U.S. Pat. No. 6,230,044; U.S. Pat. No. 6,319,199; U.S. Pat. No. 6,379,920; and WO 99/66830. The technique suitable for the diagnosis of ear-related diseases, such as otitis media, is disclosed in WO 02/39874 assigned to the assignee of the present application.

Optical measurements on the patient's ear employ an optical probe such as an otoscope, in which the ear canal is illuminated via a suitable light source. The physician can then view the image directly via an eyepiece mounted to the otoscope, or via a video image, as in U.S. Pat. No. 5,919,130 or U.S. Pat. No. 5,363,839. For reasons of sterility or hygiene and convenience, it is usually appropriate to cover the optical probe with a removable sheath or speculum that prevents contamination to or from the probe, and thus enables the same probe to be used with many patients without the need for sterilizing or disinfecting the probe itself between patients. Typically, the sheath is disposable, and thus made from a low-cost material, thus avoiding the need to sterilize or disinfect the sheath itself after use.

In more advanced systems it may be desirable to improve the quality of the light that is captured from the tissue being investigated, for example the ear canal or the vaginal walls, for the purpose of enhancing the sensitivity of subsequent analyses on this light. In WO 00/74556, for example, an optical probe is provided having an accessory device comprising an integral light-focusing element that enhances the light transmitting functions of the probe, and a window may be provided that acts as an objective for the probe's illumination elements. The accessory device may comprise optical elements such as a system of internal mirrors coupled to the window, or a toroidal ring segment in the form of an annular lens, that allow the device to act as a waveguide to direct light onto target tissues. While the light focusing effect achieved by these arrangements indirectly enhances visualization or data collection, it does not provide a simple and effective mechanism to maximize the quality of transmission of the light reflected or refracted by the tissues via the accessory device itself.

### SUMMARY OF THE INVENTION

There is a need in the art to facilitate optical measurements on the patient's body, especially for determining the patient's ear condition, by providing novel optical method and system that enable automatic identification of the patient's ear condition, such as the existence of otitis media, serous otitis media (SOM) or acute otitis media (AOM).

SOM is the medical term for accumulation of fluid within the middle ear cavity. When the fluid occurs, there usually is hearing loss, and there may be a feeling of fullness and pain. SOM often follows an upper respiratory infection, and it is much more common in children four years of age and younger. AOM is the medical term for ear infections. AOM is an inflammation of the middle ear, often accompanied by viral upper respiratory infection.

The technique of the present invention provides for detecting the existence of SOM or AOM condition, and for distinguishing between these conditions. Either one or both of the SOM and AOM conditions can be detected as a condition of the existence of fluid (mainly water) in the region of interest (ROI). As for distinguishing between the SOM and AOM, this can be based on a difference in the fluid density at the SOM and AOM conditions of the ear. The SOM-related fluid is a transparent glue or clear fluid, while the AOM-related fluid is opaque for visible-range light and therefore is relatively highly scattering for the I.R range. Hence, the fluid which accumulates in the middle ear differently scatters the propagating light, thus a different amount of light is detected at the SOM and AOM conditions. Another way to differentiate between SOM and AOM is by detecting a change in the hemoglobin level in the ROI.

The term “region of interest” or “ROI” used herein with respect to this specific application of detecting SOM and AOM, refers to a region including a middle ear cavity between the tympanic membrane and the external inner ear wall (terned “promontory”), as well as the tympanic membrane and the promontory.

According to one broad aspect of the present invention, there is provided a measurement system for use in detecting a predetermined condition of a patient's ear indicative of a certain disease, the system comprising:
(a) an optical measuring unit configured and operable for irradiating a region of interest in the patient's ear with incident light including at least two different wavelengths, detecting light responses of the region of interest to said at least two different wavelengths, and generating measured data indicative thereof, said at least two different wavelengths being selected such that the light response of the region of interest to at least one first wavelength is substantially independent of said predetermined condition and the light response to at least one second wavelength is affected by said predetermined condition; and
(b) a control unit configured and operable for controlling operation of the optical measuring unit, and for receiving the measured data and processing it to generate output data indicative of whether or not said predetermined condition exists, the control unit comprising a memory utility for storing predetermined reference data indicative of the light response of the region of interest while in a healthy condition of the ear; a data processing and analyzing utility preprogrammed for processing and analyzing the measured data by determining a relation between the measured light responses and the corresponding reference data.

The light response substantial independence of the condition to be detected means that the incident light wavelength is either substantially absorbable by the region of interest (or at least a part thereof) or substantially transmittable by the region of interest, irrespective of the absence/presence and/or change in concentration of substance(s) that are indicative of the predetermined condition. The light response detectable dependence on the predetermined condition of the ear means that the incident light wavelength is differently absorbable/transmittable or scattered by the region of interest when certain substance(s) exists or its concentration has changed in the region of interest, as compared to a normal (healthy) condition when there is no such substance or its concentration is normal in the ROI.

It should be understood that the terms “substantially absorbable” and “substantially transmittable” may and may not signify full absorption or full transmission of the specific wavelength, but rather are relative terms meaning that the specific wavelength is relatively higher absorbed or transmitted by the ROI as compared to other wavelengths.

The at least two wavelengths thus include the at least one first reference wavelength and the at least one second operating wavelength. The reference wavelength is either substantially absorbable or substantially transmittable by the ROI irrespective of whether a specific substance exists or its concentration has changes in the ROI. The reference wavelength is thus in at least one of the following wavelength ranges: about 700-900 nm and about 1420-1480 nm. The at least one operating wavelength is differently absorbable/transmittable or scattered by the region of interest when certain substance(s) exists or its concentration has changed in the region of interest. The operating wavelength is in at least one of the following ranges: about 1200-1400 nm and 1500-1700 nm. This allows for detecting a serous otitis media (SOM) condition of the patient's ear; and allows detection of AOM condition by detecting a change in scattering of the operating wavelength (increased scattering or lower intensity of the detected light) from that of the SOM condition.

Preferably, the wavelengths also include at least one additional second wavelength in at least one of the following wavelength ranges: about 540-550 nm and 570-580 nm, the system being therefore operable to detect an acute otitis media (AOM) condition via detection of a change in the hemoglobin level.

The reference data may be indicative of a relation between the light responses of the healthy ear to the at least two different wavelengths. The measured data is in the form of a relation between the light responses of the region of interest in the patient's ear to the at least two different wavelengths.

The reference data may be indicative of the light response for the operating wavelength as a function of the light response for the reference wavelength corresponding to the healthy condition. The control unit is configured and operable to process the measured data to determine the light response for the operating wavelength as a function of the light response for the reference wavelength, I^{(W)}_{λoper}=f₁(I_{λref}), and determine a difference between the reference and measured data indicative of whether fluid media exists in the region of interest being therefore indicative of the SOM condition.

The reference data may be indicative of the light responses for the second operating wavelengths as functions of the light response for the reference wavelength corresponding to the healthy condition. The control unit operates to process the measured data to determine the light response for the second operating wavelength as a function of the light response for the reference wavelength, I^{(W)}_{λoper}=f₁(I_{λref}), and the light response for the additional second operating wavelength as a function of the light response for the reference wavelength, I^{(W)}_{λoper}=f₂(I_{λref}), and determine differences between the reference and measured data indicative of whether fluid media exists in the region of interest and whether there is a change in the hemoglobin concentration as compared to that of the healthy condition, being thereby indicative of the AOM condition.

According to another broad aspect of the present invention, there is provided a measurement system for use in determining a patient's condition, the system comprising:
(a) an optical measuring unit operable for applying spectral measurements to the region of interest in a patient's body with predetermined light spectrum and producing measured spectral data indicative thereof; and
(b) a control unit for receiving and processing the measured data to generate output data indicative of the measurement results, the control unit comprising a memory utility for storing predetermined reference data representative of a value or a range of values for at least one predetermined measurable parameter corresponding to a healthy condition of a patient; a data processing and analyzing utility preprogrammed for processing and analyzing the measured data by selecting a certain part of the measured data within at least one range of the predetermined light spectrum and applying a predetermined model to the selected part of the measured data to determine a corresponding value of said at least one predetermined measurable parameter for the measured patient and to generate said output data indicative of association between the determined parameter value and the reference data.

Preferably, the processing of the measured spectral data comprises normalizing the measured spectral data to thereby obtain a relative spectrum. The predetermined model is then applied to the relative measured spectrum.

The normalization of the measured spectral data includes normalization by a reference spectrum, and preferably also normalization by a certain wavelength from the predetermined light spectrum. The result of normalizing the measured data by the reference spectrum is a normalized reflectivity spectrum.

The reference spectrum is indicative of the light intensity illuminating the region of interest as a function of wavelengths of said predetermined incident light. Generally, this can be implemented by operating the measuring unit to apply spectral measurements to a highly reflective (preferably highly diffusedly reflective) surface. Preferably, this is achieved by appropriately configuring the measuring unit, for example, by providing a plug that has a highly diffusedly reflective surface and is mounted on the measuring unit such that it is shiftable from its operative position when said surface is located in the optical path of light propagating through the measuring unit and an inoperative position of the plug when said surface is out of the optical path of said light. Hence, the measuring unit can be operated to selectively obtain the reference spectrum or the measured data.

Generally, the at least one selected range of the predetermined light spectrum is defined by the patient's condition to be detected. For example, for the purposes of determining the existence of otitis media condition in the patient's ear, the predetermined light spectrum is preferably within 300-1100 nm. The selected spectrum preferably includes a range of 500-650 nm, and/or a range of 800-950 nm.

The data processing with the predetermined model preferably includes: applying a Likelihood Algorithm to the relative measured spectrum, calculating a feature vector as a function of wavelength within the selected range, and calculating a log-likelihood ratio between the feature vector of the relative measured spectrum and that of the reference data. This ratio is scalable to determine the at least measurable parameter indicative of the patient's condition. Preferably, the control unit is configured as an expert system capable of periodically analyzing the calculated measurable parameters and optimizing the model accordingly.

Preferably, the processing of the relative measured spectrum allows for determining two measurable parameters indicative of the existence in the patient's ear of, respectively, serous otitis media (SOM) and acute otitis media (AOM).

The normalizing of the measured spectral data by the reference spectrum may be carried out by presenting the measured spectrum Eⱼ(λ,t) and the reference spectrum Wⱼ(λ,t) as, respectively,
Eⱼ(λ,t)=AIⱼ(λ,t)R_{E}(λ)Dⱼ(λ,t)andWⱼ(λ,t)=BIⱼ(λ,t)R_{w}(λ)Dⱼ(λ,t)
wherein j is the number of the measuring unit, t is the time, λ is the wavelength of incident light, A and B are unknown amplitudes, Iⱼ(λ,t) is the illumination spectrum of light source for the measuring unit j; Dⱼ(λ,t) is the light response spectrum of a detector assembly of for measuring unit j; R_{E}(λ) is the reflectivity spectrum of the region of interest; and R_{w}(λ) is the reflectivity of a reference surface used in obtaining said reference spectrum, the normalized reflectivity spectrum being thus determined as:
R(λ)=Eⱼ(λ,t)/Wⱼ(λ,t)=CR_{E}(λ)/R_{w}(λ),
wherein parameter C is a light signal amplitude depending inter alia upon a signal integration time and a distance between the measuring unit and the region of interest.

The normalized reflectivity spectrum can be further normalized by a certain wavelength λ₀ within the selected spectrum range, such that all the light intensities are measured relative to the intensity at wavelength λ₀. Hence, the effect of parameter C can be eliminated. This can be implemented by setting a relative spectrum r(λ)=R(λ)/R(λ₀) so that r(λ₀)=1. The selected value of λ₀ is the center of the wavelength range of the predetermined incident light.

Preferably, the creation of the reference data and the model includes sampling a spectrum r(λ) at certain discrete wavelengths, to generate a feature vector r={ r(λₙ),n=1, 2 . . . N }; learning probability densities f (r |A) and f (r |B) for populations including (A) healthy ears and (B) infected ears; and defining the value or range of values as a threshold T1 chosen to achieve a desired level of sensitivity corresponding to the probability of correctly diagnosing the existence of the predetermined condition of the patient's ear. The probability densities may for example include Gaussian probability densities f(r|A)=g(r, μ_{A}, P_{A}) and f(r|B)=g(r,μ_{B},P_{B}), wherein g(r,μ,P)=[2πdet(P)]^{31 N/2}exp[−½(r−μ)^{T}P⁻¹(r−μ)], μ=mean(r),P=covariance(r)=N×N matrix. The measured feature vector is then processed to determine the log-likelihood ratio as

$$\begin{matrix}L 1 ( \underline{x} ) = 2 log \{ f ( \underline{x} \vert B ) / f ( \underline{x} \vert A ) \} \\ = {( \underline{x} - {\underline{μ}}_{A} )}^{T} {P}_{A}^{- 1} ( \underline{x} - {\underline{μ}}_{A} ) - {( \underline{x} - {\underline{μ}}_{B} )}^{T} {P}_{B}^{- 1} ( \underline{x} - {\underline{μ}}_{B} )\end{matrix}$$

Then, the association between this ratio and the predetermined threshold value T1 is determined which is indicative of the existence of the otitis media in the patient's ear.

The technique of the present invention provides for identifying whether the otitis media includes serous otitis media (SOM) or acute otitis media (AOM). To this end, the creation of the reference data and the model includes defining the value or range of values as a threshold T2 chosen to achieve a desired level of sensitivity corresponding to the probability of correctly diagnosing the existence of (B1) the serous otitis media (SOM) and (B2) acute otitis media (AOM). The measured feature vector is processed to determine the log-likelihood ratio as:

$$\begin{matrix}L 2 ( \underline{x} ) = 2 log \{ f ( \underline{x} \vert {B}_{2} ) / f ( \underline{x} \vert {B}_{1} ) \} \\ = {( \underline{x} - {\underline{μ}}_{B 1} )}^{T} {P}_{B 1}^{- 1} ( \underline{x} - {\underline{μ}}_{B 1} ) - {( \underline{x} - {\underline{μ}}_{B 2} )}^{T} {P}_{B 2}^{- 1} ( \underline{x} - {\underline{μ}}_{B 2} ) ,\end{matrix}$$

and then the association between the ratio L2 and the predetermined threshold value T2 is determined being indicative of whether the detected otitis media is SOM or AOM.

Additionally, the technique of the present invention allows for conducting qualitative measurements at the same time as allowing the user (physician) to observe the target tissue itself. This is implemented by configuring the measuring unit (an optical probe) for transmitting light emanating from a target tissue (region of interest) along at least two separate optical channels. The probe comprises a probe head and a speculum member removably fitted to a distal end of the probe head. The probe head comprises light transmission means for directing an illuminating light to said target tissue via a distal end of the speculum, and means for directing light emanating from the target tissue along at least two separate optical channels. The speculum member is adapted for positioning the distal end thereof proximate to the target tissue. The distal end of the speculum member comprises an optical aperture for enabling illuminating light and emanating light to pass therethrough from and to the optical probe. The at least two separate optical channels comprise a first channel for enabling qualitative analysis of said light emanating from said target tissue, and a second channel for enabling quantitative analysis of said light emanating from said target tissue.

Preferably, the speculum member comprises an internal reflecting mirror for directing illuminating light from the light transmission means to said distal end. The probe head comprises a beam splitter arrangement for splitting light traveling in a proximal direction from said distal end into said first channel and said second channel. The term “beam splitter arrangement” refers herein to any optical arrangement capable of splitting a light beam into at least two beams, i.e., two channels or directions, substantially unaffecting the intensity or wavelength of the light. Preferably, the beam splitter arrangement comprises a parabolic mirror having an aperture therein. Alternatively, the aperture may be replaced with a plug of transparent material, which preferably non-diffracting. The aperture is configured for directing a first portion of light traveling from said distal end therethrough along the first channel and towards an objective. The latter may comprise an eyepiece ocular, or a suitable camera means for recording said image. The parabolic mirror may comprise an optical focusing element for directing a second portion of said light traveling from said distal end along said second channel and towards a light sensor.

Alternatively, or additionally, the speculum member may comprise a suitable first waveguide for directing illuminating light from said light transmission means to said distal end. The first waveguide is in the form of a first layer of material having waveguiding properties comprised in said speculum member, and the first layer having a transmitting face proximate to said distal end, and a first mating face in optical communication with said transmitting face and adapted for enabling illumination light from said light transmission means to pass therethrough to said transmitting face when said speculum member is fitted to said probe head. The light transmission means may comprise a second mating face configured to provide optical communication between said light transmission means and said first mating face when said speculum member is fitted to said probe head.

Preferably, the speculum member is disposable after use with one patient.

The speculum member preferably further comprises a plug removably fitted to said distal aperture, said plug configured to diffusedly reflect incident light thereon from said light transmission means in a known manner. The plug attachment to the speculum may be such that after shifting it into an inoperative position to be out of the optical path, it cannot be returned into the operative position, thus requiring replacement of the entire speculum by a new one.

The present invention thus provides an improved optical probe which enables qualitative measurements to be taken in parallel to enabling observation of the tissue. The probe improves the quality of transmission therethrough of the light reflected or refracted by the tissues. The probe is relatively simple in construction and simple to use, and is relatively inexpensive to manufacture.

Thus, according to yet another broad aspect of the present invention, there is provided an optical probe for transmitting light emanating from a target tissue along at least two separate optical channels, comprising a probe head and a speculum member removably fitted to a distal end of said probe head, wherein:

said probe head comprises light transmission means for directing an illuminating light to said target tissue via a distal end of said speculum, and means for directing light emanating from said target tissue along at least two separate optical channels; and wherein

said speculum member is adapted for positioning said distal end thereof proximate to the target tissue.

According to yet another aspect of the invention, there is provided a measurement system for use in determining the patient's condition, the system comprising an optical measuring unit operable for carrying out spectral measurements, the measuring unit comprising a light source system for generating light of predetermined wavelengths, a detector for collecting light impinging thereon and generating data indicative thereof, said measuring unit comprising a plug that is shiftable between its operative and inoperative positions so as to be, respectively, in and out of the optical path of light propagating from the light source system and having a highly diffusedly reflective surface, the measuring unit being selectively operable to apply spectral measurements to said surface and obtain reference spectrum data indicative of the reflectance of incident light from said surface and to apply spectral measurements to the region of interest on patient's body to obtain measured spectral data indicative of the reflectance of the incident light from the region of interest.

The system comprises or is associated with a control unit for receiving and processing the measured data to generate output data indicative of the measurement results. The control unit comprises a memory utility for storing predetermined reference data representative of a value or a range of values for at least one predetermined measurable parameter corresponding to a healthy condition of a patient and for storing the reference spectrum; a data processing and analyzing utility preprogrammed for processing and analyzing the measured data by
selecting a certain part of the measured data within at least one range of the predetermined light spectrum and normalizing said selected part of the measured data by the reference spectrum to thereby obtain a relative measured spectrum;
applying a predetermined model to said relative measured spectrum to determine a corresponding value of said at least one predetermined measurable parameter for the measured patient and generate said output data indicative of association between the determined parameter value and the reference data.

According to yet another aspect of the invention, there is provided a method for processing spectral measured data to enable determination of a patient's condition, the method comprising processing the spectral measured data indicative of reflection of predetermined incident light from a region of interest as a function of wavelengths of the incident light; said processing comprising selecting a predetermined part of the measured spectral data corresponding to at least one range of the predetermined incident light, normalizing the selected measured data to obtain a relative spectrum, and applying a predetermined model to the relative spectrum to determine a corresponding value of at least one predetermined measurable parameter and to generate output data indicative of association between the determined parameter value and preset reference data, said reference data being representative of a value or a range of values for said at least one predetermined measurable parameter corresponding to a healthy condition of a patient.

According to yet another aspect of the invention, there is provided a method for use in detecting an SOM or AOM condition of a patient's ear, the method comprising illuminating a region of interest in the ear by at least two wavelengths, detecting light responses of the region of interest to said at least two different wavelengths, and generating measured data indicative thereof, said at least two different wavelengths being selected such that the light response of the region of interest to at least one first wavelength is substantially independent of said condition and the light response to at least one second wavelength is affected by said condition.

According to yet another aspect of the invention, there is provided a method for use in detecting an SOM or AOM condition of a patient's ear, the method comprising illuminating a region of interest in the middle ear by at least two wavelengths, detecting light responses of the region of interest to said at least two different wavelengths, and generating measured data indicative thereof, said at least two different wavelengths being selected such that at least one first wavelength satisfies at least one of the following: is substantially absorbable by water or is substantially transmittable by water the light response to said first wavelength being therefore substantially independent of said condition, and the at least one second wavelength being partially absorbable by water the light response to said at least one second wavelength being therefore affected by said condition.

According to yet another aspect of the invention, there is provided a method for use in detecting an SOM or AOM condition of a patient's ear, the method comprising illuminating a region of interest in the middle ear by at least three wavelengths, detecting light responses of the region of interest to said at least three different wavelengths, and generating measured data indicative thereof, said at least three different wavelengths being selected such that at least one first wavelength satisfies at least one of the following: is substantially absorbable by water and substantially non-absorbable by hemoglobin, and is substantially transmittable by water and substantially non-absorbable by hemoglobin; the light response of the region of interest to said at least one first wavelength being therefore substantially independent of said condition, the at least two second wavelengths including a wavelength that is partially absorbable by water and a wavelength that is relatively highly absorbable by hemoglobin the light response to said at least two second wavelengths being therefore affected by said condition.

According to yet another aspect of the invention, there is provided a method for use in detecting an SOM or AOM condition of a patient's ear, the method comprising illuminating a region of interest in the middle ear by at least two wavelengths, detecting light responses of the region of interest to said at least two different wavelengths, and generating measured data indicative thereof, said at least two different wavelengths being selected such that at least one first wavelength satisfies at least one of the following: is substantially absorbable by water or is substantially transmittable by water, the light response to said first wavelength being therefore substantially independent of said condition, and the at least one second wavelength being partially absorbable by water the light response to said at least one second wavelength being therefore affected by said condition, a change in the intensity of the detected light of said at least one second wavelength from a corresponding intensity for a healthy condition being indicative of the SOM or AOM condition, and a decrease in the intensity of the detected light to said at least one second wavelength from that corresponding to the SOM condition being indicative of the AOM condition.

According to yet another aspect of the invention, there is provided a method for use in determining a patient's condition, the method comprising:
(i) providing reference data representative of a value or a range of values for at least one predetermined measurable parameter corresponding to a healthy condition of a patient, and a certain reference spectrum corresponding to reflectance of a predetermined light spectrum from a reference highly reflective surface;
(ii) applying spectral measurements to a region of interest on the patient's body with predetermined light spectrum and producing measured spectral data indicative thereof; and
(iii) processing the measured data to generate output data indicative of the measurement results, said processing comprising selecting a part of the measured data within at least one range of the predetermined light spectrum and applying a predetermined model to the selected part of the measured data to determine a corresponding value of said at least one predetermined measurable parameter for the measured patient and generate said output data indicative of association between the determined parameter value and the reference data.

### BRIEF DESCRIPTION OF THE DRAWINGS

In order to understand the invention and to see how it may be carried out in practice, preferred embodiments will now be described, by way of non-limiting example only, with reference to the accompanying drawings, in which:

FIG. 1A exemplifies a block diagram of an optical probe according to the invention;

FIG. 1B illustrates the water transmission spectra for the depth of water of 0.3 mm and 5 mm, theoretically calculated from the water absorption coefficient spectra;

FIG. 1C shows the experimental results of using the technique of the present invention for detecting the SOM condition;

FIG. 1D illustrates the simulation results of using the technique of the present invention for determining the AOM condition;

FIGS. 1E and 1F exemplify a fibers' arrangement suitable to be used in the probe of FIG. 1A;

FIG. 2 is an isometric sectional side view of an optical probe according to another example of the invention;

FIG. 3 is a cross-sectional side view of an optical probe according to yet another example of the invention;

FIG. 4 shows in greater detail the distal end of the probe of FIG. 3;

FIG. 5 schematically illustrates an optical measurement system according to the invention for use in determining the condition of a patient's ear;

FIGS. 6A and 6B show two examples, respectively, of a method according to the invention;

FIGS. 7A and 7B exemplify a calibration step showing, respectively, the ear spectrum (measured spectral data) and white spectrum (reference spectral data);

FIGS. 8A and 8B show experimental results of another example of the method of the present invention for determining the otitis media in the patient's ear.

### DETAILED DESCRIPTION OF THE INVENTION

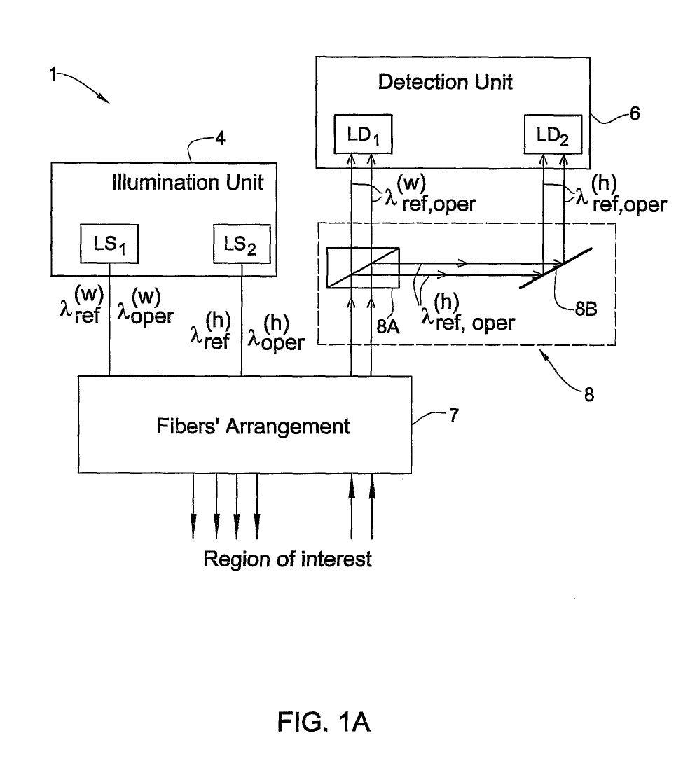

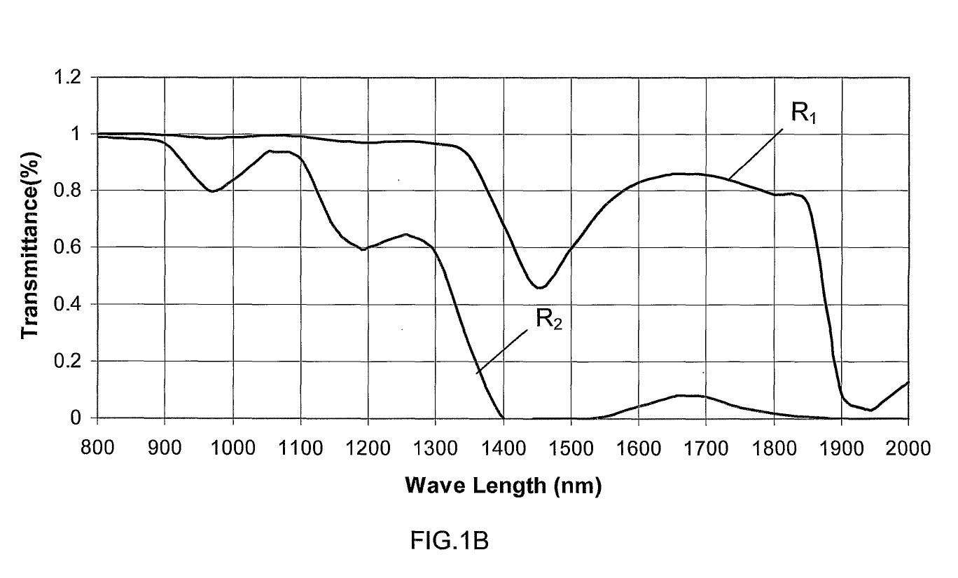

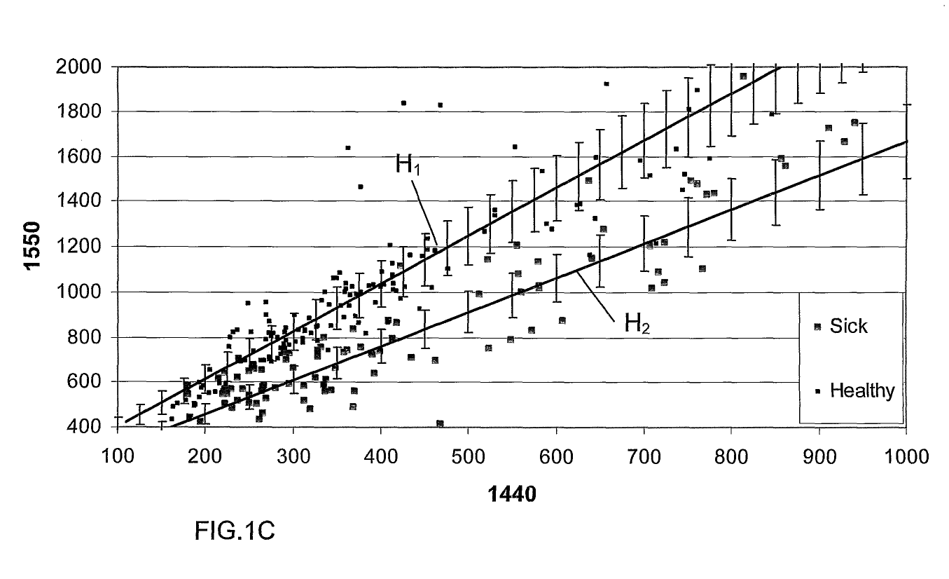

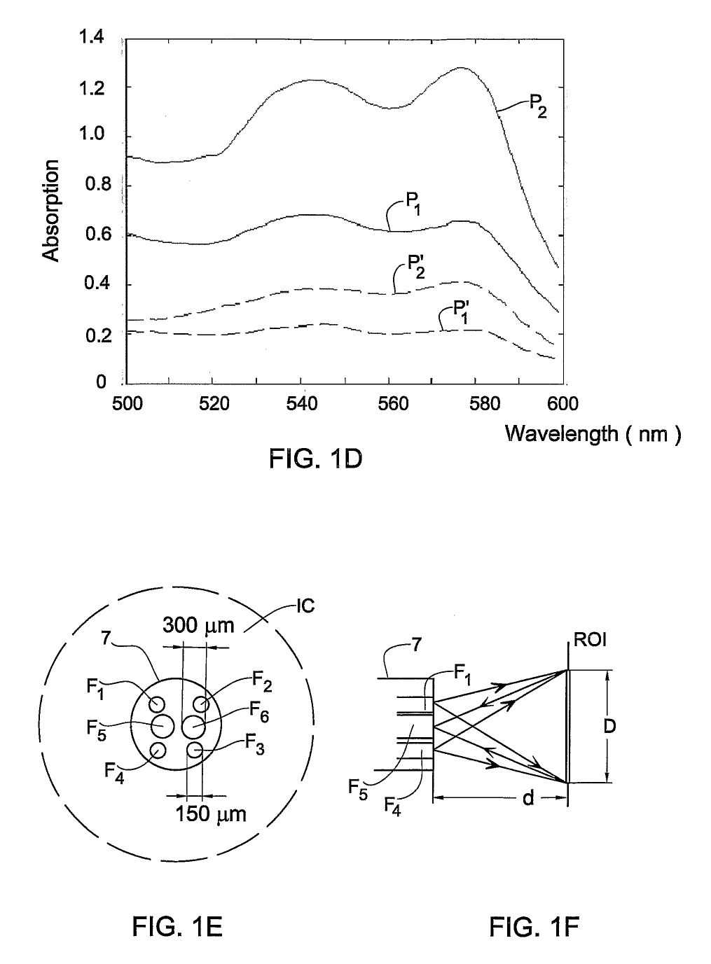

The present invention in its one aspect relates to an optical probe for measuring in a patient's ear. FIG. 1A is a block diagram of an optical probe 1 configured as an otoscope. The probe 1 includes an illumination unit 4 having one or more light sources—two light sources LS₁ and LS₂ in the present example; a detection unit 6 having one or more light detectors—two light detectors LD₁, and LD₂ in the present example; and a light directing assembly 7 including one or more optical fibers—six fibers in the present example. Also optionally provided in the light directing assembly is a filtering unit 8 accommodated in the optical path of light emerging from the fibers and propagating towards the detectors and configured for separating between light portions of different wavelengths. The filtering unit 8 includes one or more spectral filters (e.g., dichroic beam splitter, grating, resonator filter). In the present example, the filtering unit 8 includes a dichroic mirror 8A, and also a mirror 8B the provision of which is optional (the second detector LD₂ may be located in the optical path of light reflected from the dichroic mirror 8A).

It should be noted that, generally, the illumination unit and/or the detection unit and/or the light directing assembly may be mounted inside the common housing; or the light sources and/or detectors may be mounted outside the housing and be connected to the inside of the housing via the fibers.

In the present example, the probe 1 is configured and operable to monitor SOM and AOM conditions. In order to monitor the SOM or AOM condition, the probe is configured to detect the existence of fluid media within the middle ear region, constituting a region of interest (ROI). In order to distinguish between the SOM and AOM conditions, the probe is configured to detect a change in the hemoglobin concentration level in the Tympanic membrane. In this connection, the term “middle ear” refers to a region including either one or both of the tympanic membrane, and the middle ear cavity between the Tympanic membrane and the Promontory.

Generally, according to the invention, in order to monitor such condition(s) of the ROI as the existence and/or a change in concentration (e.g., over-concentration) of a certain substance in the ROI, the illumination unit of the present invention is configured and operable to produce light including a wavelength or wavelength range selected such that a light response of the ROI to this wavelength depends on the predetermined condition of the ROI. Preferably, however, in order to calibrate the device and in order to take into account various tissue-associated effects, the illumination unit is configured to produce light including at least two different wavelengths or wavelength ranges. At least one of these wavelengths, termed “reference wavelength ”, is selected such that a light response of the ROI to this wavelength (reflection of the ROI) is substantially independent of the predetermined condition (i.e., of whether the specific substance exists in the ROI and/or the concentration of a specific substance in the ROI has changed from a normal value or range of values). This means that the ROI or at least a part thereof either substantially absorbs or substantially transmits the reference wavelength, irrespective of presence/absence or a change in a concentration of the substance of interest in the ROI. At least one other wavelength, termed “operating wavelength ”, is selected such that the light response of the ROI to this operating wavelength (reflection) depends on the predetermined condition of the ROI. This means that existence of certain substance or a change of the concentration of the substance of interest affects the absorption/reflection of the operating wavelength. It should be noted that the term “reflection” used herein refers to both specular reflection and scattering.

It should be understood that in order to produce such at least two different wavelengths, either a single light source is used producing broadband illumination including the required wavelengths, or separate light sources are used each for generating the required wavelength(s).

In order to monitor such a condition as the existence of fluid media (mainly water) in the middle ear, the reference wavelength λ^{(W)}_{ref} and operating wavelength λ^{(W)}ₒₚₑᵣ are preferably selected to be, respectively, of about 1420-1480 nm or 700-900 nm, and of about 1200-1400 nm or 1500-1700 nm.

The reference wavelength range of 1420-1480 nm is highly absorbable by water and is thus highly absorbable by the tympanic membrane which typically includes 70-80% of water. Hence, the illuminating reference wavelength of this range, e.g., 1440 nm, while propagating towards the middle ear cavity is highly absorbed by the tympanic membrane and substantially does not reach the middle ear cavity therebehind; and thus the detected reflection is independent of whether water exists in the middle ear cavity or not. Even if a certain portion of incident light passes through the tympanic membrane (since the water absorption/transmission spectrum typically depends on the depth of water), this light portion is then highly absorbed by promontory which also includes water. These affects are irrespective of whether water exists in the middle ear cavity or not. The other possible reference wavelength range of 700-900 nm is substantially transmitted by water. Hence, the illuminating reference wavelength, e.g., 830 nm, is substantially transmitted by the tympanic membrane and through the middle ear cavity irrespective of whether it contains water or not, and is partially reflected by the promontory; the detected reflection is thus independent of whether water exists in the middle ear cavity or not.

The operating wavelength range (1200-1400 nm; or 1500-1700 nm) is partially absorbable by water. Hence, such wavelength (e.g., 1550 nm) while propagating towards the middle ear cavity is partially absorbed by the tympanic membrane, then partially absorbed by water, if any, in the middle ear cavity, and then reflected by the promontory. Hence, if water exists in the middle ear cavity (AOM or SOM condition), then the detected light intensity is decreased as compared to that of no water in the middle ear cavity (healthy condition). The detected reflection of the operating wavelength is thus indicative of the AOM or SOM condition.

FIG. 1B shows the water media transmission spectra (transmission as a function of wavelength), where graphs R₁ and R₂ correspond to the depth of water of, respectively, 0.3 mm and 5 mm. These functions are calculated theoretically from the water absorption coefficient spectra. Graph R₁ demonstrates that with the incident light of reference wavelength (1420-1480 nm) certain amount of incident light might pass thorough the tympanic membrane (which is of about 0.3 m thickness). But, as indicated above, this light will be then highly absorbed by the promontory which also contains high amount of water (about 70-80 percent of water).

FIG. 1C shows the experimental results of applying the device of the present invention to numerous “healthy” and “AOM or SOM sick” patients. Two graphs H₁ and H₂ are shown, each presenting, in relative units, the intensity of detected light for 1550 nm wavelength (operating wavelength) as a function of the intensity of detected light for 1440 nm wavelength (reference wavelength); each point or group of point corresponding to measurement session (one or more measurements) as applied to a specific patient. Graphs H₁ corresponds to the measurements on “healthy” patients, and graph H₂ corresponds to the measurements on “sick” patients. As also shown in the figure, a certain range of the intensity values is considered when evaluating the measurement results. The experiments thus show that the technique of the present invention provides for clearly distinguishing between the healthy and sick conditions.

Using illumination with both the reference and operating wavelengths enables estimation of a “noise” part of the detected light, which is substantially the same for the reference and operating wavelengths and can thus be extracted from measured data. This noise part is mainly associated with the light scattering at the outer surface of the tympanic membrane, which is typically a diffusing surface (since it has a small-feature surface relief), and is thus highly dependent on a distance (termed “working distance”) between the probe and the ROI. To this end, the probe is configured so as to ensure substantially the same optical path for both the reference and operating wavelengths, as will be described below.

Thus, in order to detect the AOM or SOM condition, the use of two illuminating wavelengths is sufficient. In the present example of FIG. 1A, these two wavelengths are generated by the same light source LS₁, e.g., LED, and a common InGaAs detector LD₁ is used for detecting the reflections of these wavelengths.

In order to detect the AOM condition (which differs from the SOM condition in the relatively high scattering from the fluid medium in the middle ear cavity due to impurities in water), the control unit may operate to identify a difference in the detected light intensity associated with an increased scattering. Alternatively or additionally, in order to identify the AOM condition, as well as to detect any other inflammation condition, the probe device is configured to detect a change in the hemoglobin level from that of the healthy condition. To this end, the operating wavelength λ^{(h)}ₒₚₑᵣ of about 570-580 nm (preferably 575 nm) is selected, which is relatively highly absorbable by hemoglobin (absorption peak) and thus the detected reflection of this wavelength from ROI depends on the amount of hemoglobin in the ROI. As for the hemoglobin-associated reference wavelength λ^{(h)}_{ref}, the same wavelength range as that of the water-associated reference wavelengths may be used (e.g., 1440 nm, 720 nm or 830 nm), where the light response of the ear to these wavelengths is substantially independent of changes in the ear condition, since these wavelengths are substantially non-absorbable by hemoglobin.

FIG. ID shows the simulation results in the form of light absorption as a function of wavelength. Here, graphs P₁ and P₂ correspond to, respectively, to the healthy and sick conditions, and graphs P₁ and P₂ correspond to the standard deviation for the same. The sick condition (increased concentration of hemoglobin) can thus be detected.

In the present example of FIG. 1A, the reference and operating wavelengths for hemoglobin level monitoring are generated by the same light source LS₂, e.g., LED, and the reflections of these wavelengths are detected by a silicon detector LD₂.

It should be noted that the provision of additional hemoglobin-associated reference wavelength (or wavelength range) is optional, and generally the use of three different wavelengths (or wavelength ranges) is sufficient for SOM and AOM measurements, namely one reference wavelength and two operating wavelengths. It should also be noted that as the otoscope typically defines an imaging channel for visual observation of the ROI by a physician, the same light source LS₂ may be used in the imaging and hemoglobin measurement channels.

It should also be noted that, generally, the probe may be configured for spectral measurements using one or more light sources producing light including the desired wavelengths. A spectrometric detector arrangement (using a single or multiple light detectors) may be used for detecting the light response of ROI, in which case the detected spectrum is then analyzed to select therefrom light intensities corresponding to the desired wavelengths or wavelength ranges.

Turning now to FIGS. 1E and 1F, there is schematically illustrated an example of arranging the fibers for directing illuminating light towards the ROI and for collecting reflected light and directing it towards the detection unit. As shown in FIG. 1E illustrating a cross sectional view of the distal end of the probe by which it is brought to the ROI, in this specific but non-limiting example, a fiber bundle F containing six fibers F₁-F₆ is used, four of them F₁-F₄ being illuminating fibers for transmitting four selected wavelengths λ^{(W)}_{ref}, λ^{(w)}ₒₚₑᵣ, λ^{(h)}_{ref}, λ^{(h)}ₒₚₑᵣ, respectively, from the light source(s) towards the ROI, and the other two fibers F₅ and F₆ being light collecting fibers for collecting the reflected light and transmitting it towards the detection unit. Again, it should be understood that a single illuminating fiber may be used for transmitting all the incident light wavelengths; and the provision for the second reference wavelength is optional. As also shown in the figure in dashed lines, this fiber bundle F may be placed in the central region (of about 1 mm diameter) of the probe's distal end (of about 4 mm diameter), while a periphery region of the probe, outside the fiber bundle F, serves as an imaging (visual) channel IC for the propagation of reflected light to be used by a physician to visually observe the ROI and/or to be collected by an imaging device, as the case may be.

Generally, the fibers arrangement F is such as to ensure that all the incident light portions (from all the fibers) illuminate substantially the same spot in the ROI, and the illuminating and reflected light portions propagate substantially equal optical paths between the distal end of the probe and the ROI (working distance). To this end, the numerical aperture of each of the fibers F₁-F₆ is selected in accordance with the working distance d such that the output of the illuminating fibers F₁-F₄ presents a point-like light source for the illuminated spot, and the light collection is carried out with the same numerical aperture as the illumination.

Considering that the illuminated spot diameter D is to be of about 7-8 cm, and that the working distance d is about 15 mm, the illuminating fibers F₁-F₄, each having a 100 μm core diameter, are located in a spaced apart relation so as to surround light collecting fibers F₅ and F₆ each having a 300 μm core diameter. For example, the illuminating fibers are located at the comers of a rectangle (or arranged in a circular array). The light collecting fibers are located one adjacent to the other between the illuminating fibers. The numerical aperture of the fibers is such that an overlapping region between light spots produced by light portion from the fibers within the ROI is significantly larger than a space between these spots, practically the overlapping region is of about the desired spot size D, namely of about 7-8 cm.

Turning back to FIG. 1A, it should be understood that the filtering unit 8 may include two spectral filters (e.g., dichroic beam splitters) accommodated at the output of the collecting fibers, respectively, and associated with respective light detectors.

It should also be noted that the probe may be formed with a speculum member at the distal end of the probe housing. The speculum member can be integral with the housing, or removably mountable onto the distal end of the housing, as will be exemplified further below. Referring to FIG. 1E, the peripheral region of the probe's distal end may be constituted by the speculum mounted onto the probe. The relative arrangement of the fiber bundle and the visual channel may be different from that exemplified in FIG. 1E. For example, the fibers may be located within the speculum, and the visual channel be represented by the central region of the probe.

An optical probe of the present invention may be configured for transmitting light emanating from a target tissue (constituting a region of interest) along at least two separate optical channels, and include a probe head and a speculum member removably fitted to a distal end of the probe head. The probe head includes light transmission means for directing illuminating light to the target tissue via a distal end of the speculum, and means for directing light emanating from the target tissue along at least two separate optical channels. The speculum member is adapted for positioning the distal end thereof proximate to the target tissue.

Unless otherwise stated, the term “proximal” (P) herein refers to a direction away from the target tissue and towards the user of the optical probe, while the term “distal” (D) refers to a direction towards the target tissue and away from the user.

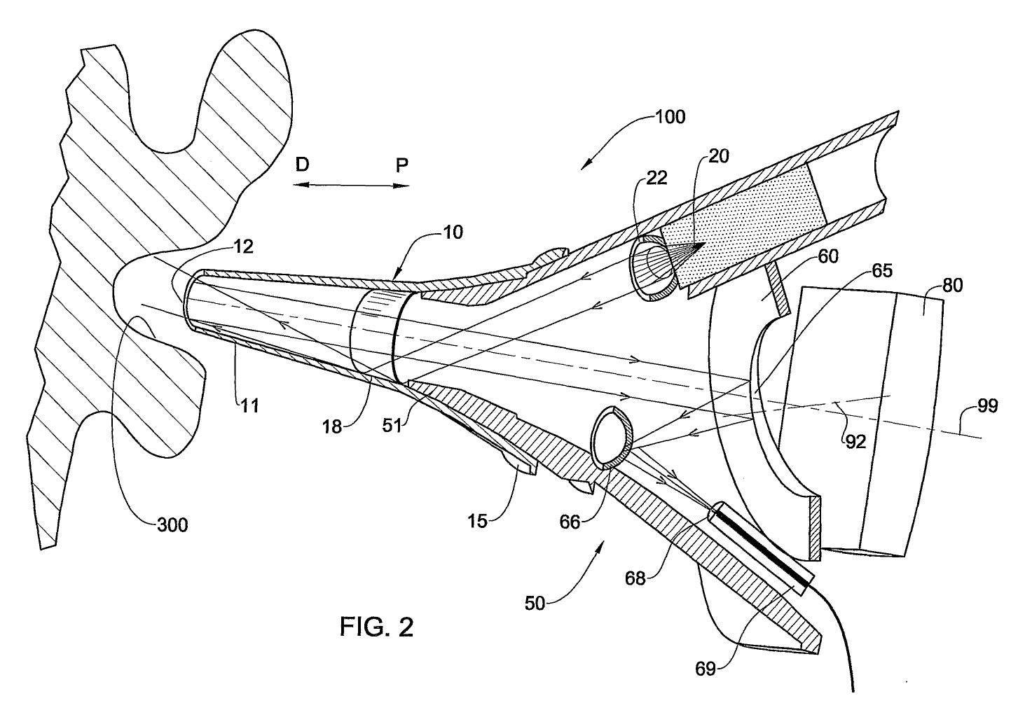

Referring to FIG. 2, another example of an optical probe, generally designated 100, is illustrated. The probe 100 includes a speculum member 10 removably fitted to a probe head 50. The speculum member 10 is generally frustoconical in form, having a smaller distal end 11 with aperture 12, and a larger proximal end 15. The speculum member 10 is hollow, enabling optical communication between the aperture 12 and the proximal end 15.

The probe head 50 is also typically frustoconical in form, the smaller distal end 51 thereof generally configured for engagement with the speculum member 10. The inner conical surface of the proximal end 15 is typically configured to provide a press-fit engagement with the outer conical surface of the distal end 51 of the probe head. Alternatively, suitable engagement means such as a bayonet fitting or complementary screw threads may be provided for removably fitting the speculum member 10 to the probe head 50.

The probe head 50 is typically hollow, having a distal end 51 which is in optical communication with aperture 12 when the speculum member 10 is engaged with the probe head 50. The probe head 50 has an objective 80 at the proximal end thereof, which may be an eyepiece ocular, for example, to permit direct visual observation of light passing through said aperture 12 along principal axis 99. Alternatively, the objective 80 may comprise any suitable video camera or CCD device, for recording images transmitted from a target tissue 300 via the aperture 12. The target tissue 300, which may be the ear canal, or vaginal walls, for example, according to the specific application of the probe, is illuminated by light produced by one or more suitable light source 20 via an appropriately provided light transmission assembly such as a focusing element 22. Alternatively, the light source is remote from the probe 100, and suitable optical fiber(s) arrangement provides optical communication between the light source and the focusing element 22. Advantageously, the light source 20 and/or the focusing element 22 are aligned with the outer wall of the probe head 50, and thus at an angle with respect to the principal axis 99. Accordingly, the speculum member 10 is provided with an internal reflector 18 configured to direct illuminating light incident thereon from said focusing element 22 towards the aperture 12 and therefrom to the target tissue 300 when this is in close proximity to the probe 100.

As a result of illuminating the target tissue 300 with light, a light response (typically reflected light) of the tissue is produced which passes into the interior of the probe 100 via the aperture 12. The probe head 50 comprises a beam splitter arrangement in the form of a parabolic mirror 60 having a central aperture 65. By means of this aperture 65, a first portion of light traveling from the distal aperture 12 along the principal axis 99 is directed towards a first optical channel and objective 80, permitting visualization of the target tissue 300 either directly or indirectly. The parabolic mirror 60 has its axis of symmetry 92 inclined to the principal axis 99 and directed towards a convex mirror 66, positioned on the inner wall of the probe head, which in turn directs incident light thereon towards objective 68. Thus, a second portion of light traveling from the distal aperture 12 parallel to the principal axis 99 is reflected by the parabolic mirror 60 and convex mirror 66 and thus directed towards a second optical channel and objective 68. A suitable light sensor 69, or alternatively a suitable optical fiber arrangement, is provided at objective 68, and provides operative communication with a suitable analysis unit (preferably a spectrometer), to enable analysis of the light received from the tissue sample.

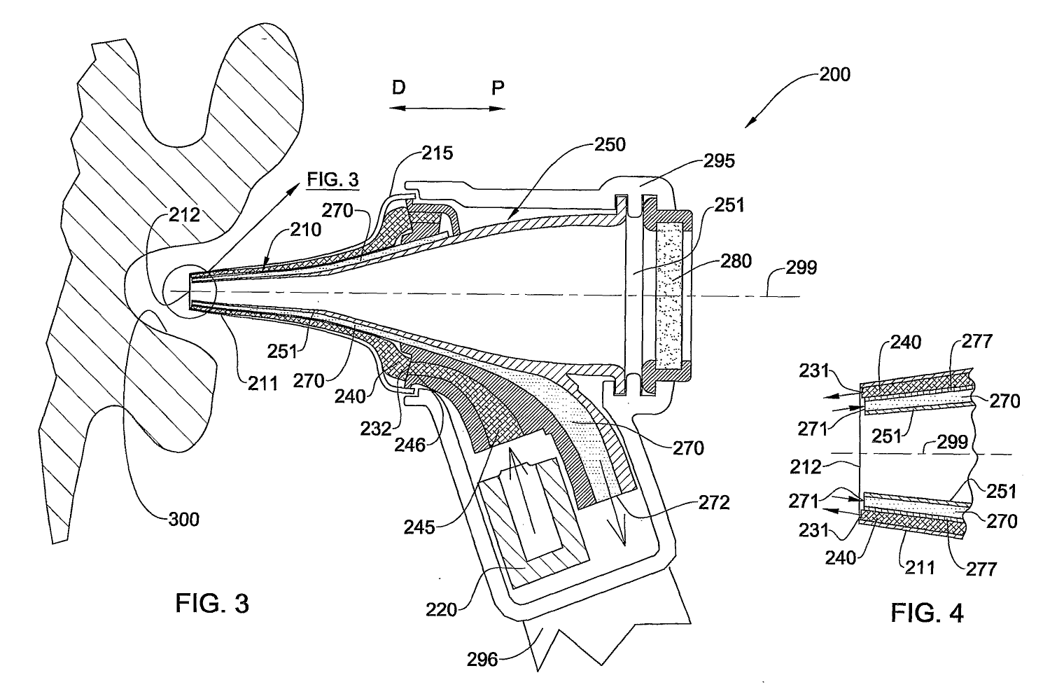

Referring to FIGS. 3 and 4, yet another example of the optical probe, generally designated 200, is illustrated. The probe 200 includes a speculum member 210 that releasably fits over the distal end 251 of the probe head 250.

As with the example of FIG. 2, the speculum member 210 is generally frustoconical in form, having a smaller distal end 211 with aperture 212, and a larger proximal end 215. The speculum member 210 is hollow, enabling optical communication between the aperture 212 and the proximal end 215.

The probe head 250 is also typically frustoconical in form, the smaller distal end 251 thereof generally configured for engagement with the speculum member 210. The inner conical surface of the proximal end 215 is typically configured to provide a press-fit engagement with the outer conical surface of the distal end 251 of the probe head. Alternatively, suitable engagement means such as a bayonet fitting or complementary screw threads may be provided for removably fitting the speculum member 210 to the probe head 250.

The probe head 250 is typically hollow, having a distal end 251 in optical communication and close proximity with aperture 212. The probe head 250 has an objective 280 at the proximal end thereof, which may be an eyepiece ocular to permit direct visual observation of light passing through said aperture 212 along principal axis 299. Alternatively, the objective 280 may comprise any suitable video camera or CCD device, for recording images transmitted from a target tissue 300 via distal aperture 212. The target tissue 300, which may be the ear canal, or vaginal walls, for example, according to the specific application of the probe, is illuminated by means of a suitable light source 220 via a suitable light transmission means. In this embodiment the light transmission means comprises a waveguide arrangement in the form of a layer 240 of material having waveguiding properties, such as for example PMMA (polymethylmetacrylate) for example, and bonded or otherwise attached to the inner surface of the speculum member 210. The waveguiding layer 240 has a transmission face 231 proximate to the aperture 212, and a distal mating face 232 in optical communication with the transmission face 231. Another waveguide 245 is provided in the probe head 250, and having a mating face 246 at lo one end thereof complementary to said mating face 232, and the other end of the waveguide 245 is connected to or connectable with the light source 220. The light source 220 may optionally be remote from the probe 200, and a suitable optic fiber arrangement provides optical communication between the light source and the wave guide 245. Thus, when the speculum member 210 is properly engaged with respect to the probe head 250, the mating faces 232 and 246 are aligned and in optical contact, enabling illumination light to be transmitted to the target tissue 300 via the transmission face 231, when the probe 200 is in close proximity to the tissue 300.

As with the example of FIG. 2, as a result of illuminating the target tissue 300 with light, a light response (typically reflected light) is produced from the tissue and passes into the interior of the probe 200 via the aperture 212. The probe head 250 is typically hollow, enabling a first portion of light traveling from said aperture 212 along the principal axis 299 to be directed towards a first optical channel and towards objective 280, permitting visualization of the target tissue 300 either directly or indirectly. In this embodiment, the second optical channel comprises a suitable second waveguide for directing light from said aperture 211 towards a light sensor. The second waveguide is in the form of a layer of material 270 having waveguiding properties comprised on the outer distal surface of the probe head 250. The said second layer 270 is typically made from PMMA or the like, for example, and has a receiving face 271 proximate to the aperture 212, and a transmitting face 272 in optical communication with the receiving face 271 and adapted for enabling light from outside of aperture 212 to pass therethrough to said second transmitting face 272. The transmitting face 272 is adapted for optical communication with a suitable light sensor, directly or indirectly, or alternatively with a suitable optical fiber arrangement, which provides operative communication with a suitable analysis unit, typically a spectroscope, enabling analysis of the light received from the tissue sample. Preferably, the layer 270 is circumferentially covered by a protective layer 277, made of for example metal or plastic, for minimizing damage to the waveguide, particularly during engagement and disengagement of the speculum member 210.

Preferably, the probe 200, and at least the probe head 250, is accommodated in a suitable housing 295, which also comprises a handle 296 to facilitate handling of the probe by the user.

In all embodiments, the speculum member is preferably disposable after one or multiple use with one patient, and is thus preferably made from a relatively inexpensive material.

Preferably, but optionally, the speculum member includes a plug (which is not specifically shown) that closes the distal aperture thereof. The plug is mounted so as to be shiftable from its operative position when it closes the aperture and thus is in the optical path of light propagating through the probe when in operation, and an inoperative position when it is out of said optical path. The plug at least at its inner surface is made from a suitable material that diffuses and reflects incident light thereon, and thus may be used by the optical probe of the present invention for calibration purposes. Thus, prior to using the optical probe with a patient, the intensity of diffused reflected light obtained via the second channel when the plug is internally illuminated by the illuminating light may be compared to the intensity of the illumination light. The ratio of intensities thus obtained is the compared with expected nominal datum values, and any deviation therefrom may then be applied to any qualitative measurements of intensities taken of the target tissues, the plug having being removed before such measurements.

In another aspect, the present invention relates to a measurement system and method for use in determining the patient's condition, in particular the ear condition. More specifically the system is used for determining whether the ear is healthy or is infected with otitis media or serous otitis media, and is therefore described below with respect to this specific application.

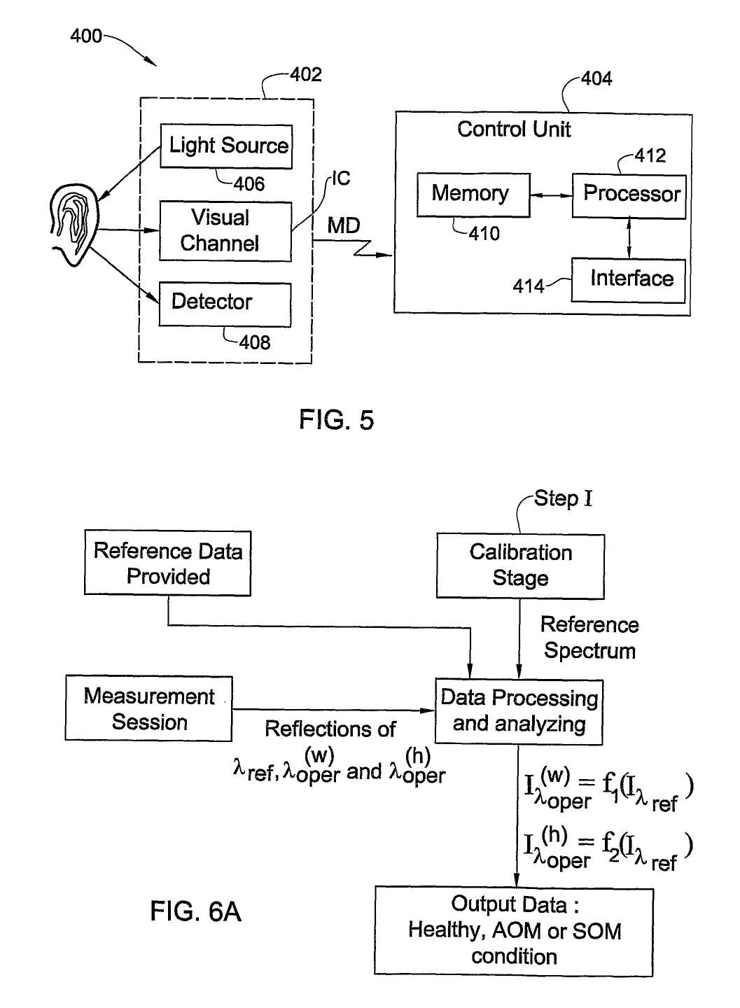

Referring to FIG. 5, an optical measurement system, generally at 400 is schematically illustrated. The system 400 is configured for determining the condition of a patient's ear. The system 400 comprises such main constructional parts as an optical measuring unit 402 for applying spectral measurements to the inside of the patient's ear; and a control unit 404 connectable to the measuring unit either via a communication cable or wireless communication means.

The measuring unit 402 is an optical probe, which may be designed as either one of the above-described examples. Generally, the measuring unit 402 includes a light source assembly (illumination unit) 406 for producing illuminating radiation of one or more predetermined wavelength range, for example 400-1600 nm; a detector assembly (detection unit) 408 for receiving light response of the illuminated region in the ear (light reflected from the illuminated region) and generating measured data indicative thereof. The optical probe also preferably includes appropriate light transmitting arrangement defining measurement channels(s) and also defining an imaging (visual) channel IC shown in the figure in dashed lines. As indicated above, light produced by the same one or more of light source elements of the illumination unit 406 may be used for the measurement and imaging channels. The detection unit 408 may be configured as a spectrometer or may include at least two detectors for detecting light of different wavelength ranges. The measuring unit preferably also includes a light directing assembly for spatially separating incident and reflected light. These may for example be optical fibers, and/or mirrors' arrangement.

The control unit 404 is typically a computer system including inter alia a memory utility 410 for storing certain reference data; a data processing and analyzing utility 412; and a user interface utility 414. The data processing and analyzing utility is preprogrammed for processing input measured data by applying thereto a predetermined mathematical model.

The reference data is obtained by performing a general learning mode, and carrying out a calibration stage prior to applying a measurement session to a specific patient. The learning mode consists of applying numerous measurements to various patients and determining measured data corresponding to healthy conditions.

According to one embodiment of the invention, the reference data includes light intensities corresponding to the light response of the healthy ear at several selected wavelengths or wavelength ranges of incident light, e.g., at least one reference wavelength and at least one operating wavelength considering that SOM condition is to be detected or at least two operating wavelengths considering that AOM is to be detected.

According to another embodiment of the invention, the reference data includes, per each disease to be detected, a value or a range of values for at least one spectral factor defining a boundary between the healthy and diseased condition. The spectral factor is determined by processing measured spectral data in the form of a relative spectrum of the ROI (relative light intensity as a function of wavelength) with the predetermined mathematical model, as will be described below.

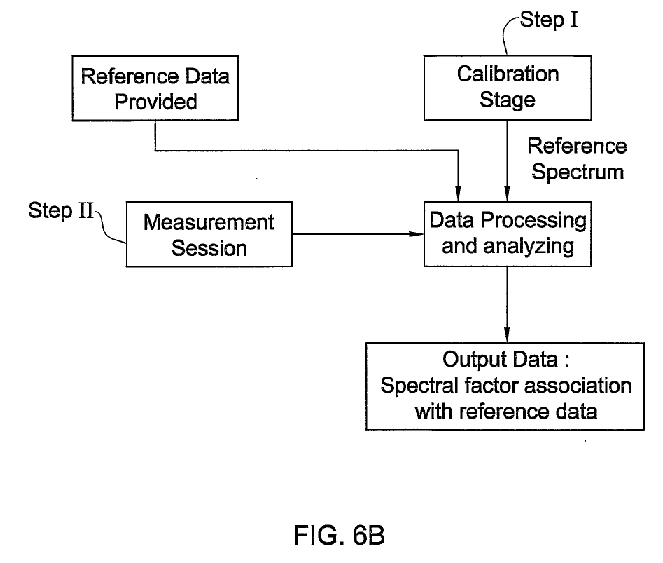

The method of the present invention will now be exemplified with reference to FIGS. 6A and 6B and FIGS. 7A-7B, 8A-8B.

FIGS. 6A and 6B show two examples, respectively, of a flow diagram of the invented method. In both examples, initially, a calibration stage is preferably carried out—Step I. Generally, the calibration stage is aimed at eliminating or at least significantly reducing the effects of variation in the light source and detector response in the measured data. Such variations exist between different instruments, and different times, even on the same instrument. At the calibration stage, a white reference spectrum, R_{w}(λ), is obtained. This can be implemented as described above, namely, by placing a highly reflective (preferably, diffusive reflective) element at the distal end of the measuring unit (in the case of otoscope, the distal end of a speculum) and operating the measuring unit to determine the reflectivity of this white surface, which is considered to be equal to the intensity of incident light reaching the region of interest.

Then, an actual measurement session (at least one measurement of a predetermine duration) is applied to the region of interest (e.g., patient's ear)—Step II. The region of interest is illuminated with predetermined incident radiation, reflected light is detected, and measured data is produced. According to the example of FIG. 6A, the actual measurement session includes illuminating the ROI with at least two different wavelengths or wavelength ranges (preferably, at least three different wavelengths) as described above with reference to FIGS. 1A-1D. The illumination is preferably carried out in pulses, e.g., 1msec pulse, and the measurement session (illumination/detection) duration is of about 100 msec. Measured data is indicative of the reflections of the ROI to the illuminating wavelengths, λ_{ref} (or two different reference wavelengths), λ^{(W)}ₒₚₑᵣ and λ^{(h)}ₒₚₑᵣ. This measured data may be derived from spectral data. According to the example of FIG. 6B, the ROI is illuminated with broadband illumination and the measured data is in the form of spectral data (the detected light intensity as a function of all the wavelengths of the incident light).

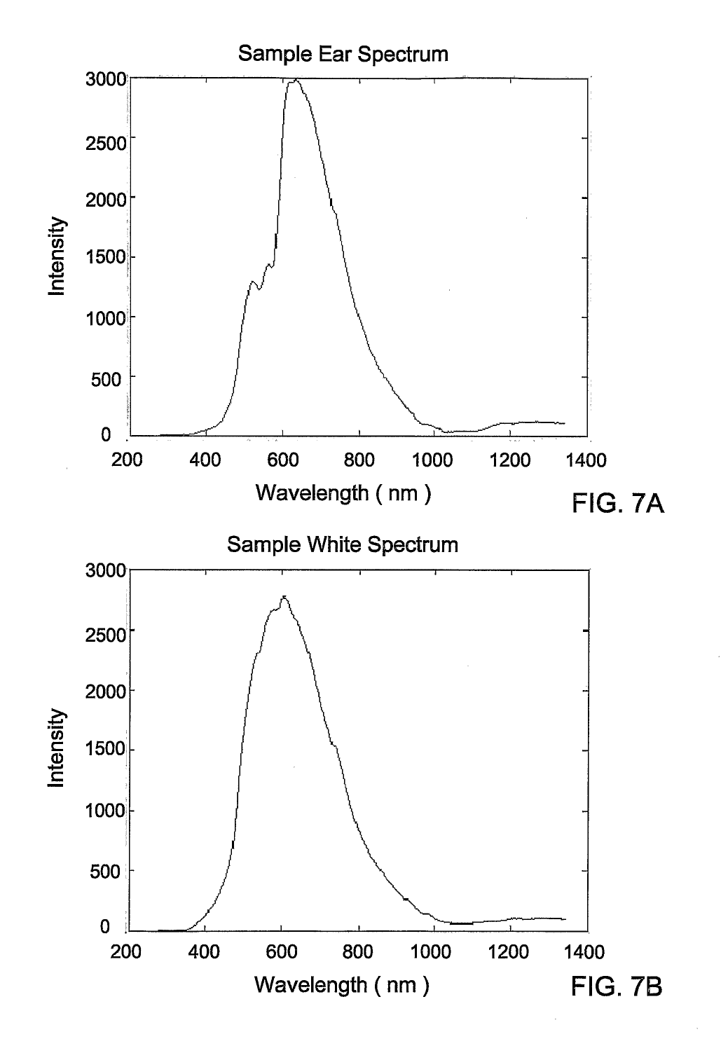

FIGS. 7A and 7B exemplify, respectively, the ear spectrum (measured spectral data) and white spectrum (reference spectral data). In the specific, but non-limiting, example of determining the patient's ear condition, incident light of 300-1400 nm is used. The white spectrum is obtained prior to applying the actual measurement session to each patient (at the calibration stage) and is stored in the memory utility. It should be noted that, generally, the calibration stage may be conducted periodically and not necessarily repeated for each new patient.

The measured data is received at the control unit where it is processed and analyzed with the predetermined mathematical model—step III.

According to the example of FIG. 6A, the processing of the measured data consists of determining, for water and hemoglobin, a relation between the detected light intensity (reflection) for the operating wavelength and the detected light intensity for the reference wavelength, namely, the light intensity for operating wavelength, I^{(W)}_{λoper}, as a function ƒ₁ of the light intensity for the reference wavelength, I_{λref}, and similarly for hemoglobin, I^{(h)}_{λoper}=f₂(I_{λref}), where the reference wavelength may be the same of different for water- and hemoglobin-related measurements. The measured intensity may be calculated as integral of the detected light pulses during the measurement session (as in the example of FIG. 1C). The output data in then generated being indicative of whether the patient's ear is classified to “healthy”, “AOM” or “SOM” condition. If the reflection of water-associated operating wavelength falls within the predetermined range of “normal” intensities (see FIG. 1C) then the patient's condition is considered as “healthy” (where “normal intensity range is defined as reference data or calibration curve); if the reflection of water-associated operating wavelength is lower than the “normal” intensity (considering a certain threshold) and the reflection of hemoglobin-associated wavelength corresponds to “normal” hemoglobin level, then the patient's condition is considered as relating to SOM; and if the reflection of water-associated operating wavelength is lower than the “normal” intensity and the reflection of hemoglobin-associated wavelength is lower than the “normal” one, then the patient's condition is considered as relating to AOM.

According to another embodiment of the invention, the processing of the measured data consists of normalizing the measured spectrum by the reference spectrum to obtain a normalized reflectivity spectrum R(λ). The normalized reflectivity spectrum is then processed to determine a corresponding value of at least one measurable parameter (the so-called “spectral factor”). The normalized reflectivity spectrum R(λ) is independent of instrument number j and of time t.

Mathematically, the normalization process can be described as follows:
Eⱼ(λ,t)=AIⱼ(λ,t)R_{E}(λ)Dⱼ(λ,t)
Wⱼ(λ,t)=BIⱼ(λ,t)R_{w}(λ)Dⱼ(λ,t)
wherein:

Eⱼ(λ,t) is the measured spectrum of the region of interest (e.g., ear);

Wⱼ(λ,t) is the measured white reference spectrum;

j is the instrument number;

t is the time;

λ is the wavelength of incident light;

A,B are unknown amplitudes;

Iⱼ(λ,t) is the illumination spectrum of light source for instrument j;

Dⱼ(λ,t) is the response spectrum of the detector for instrument j;

R_{E}(λ) is the reflectivity spectrum of the ear drum;

R_{w}(λ) is the reflectivity of a standard white surface

The normalized reflectivity spectrum is determined as:
R(λ)=Eⱼ(λ,t)/Wⱼ(λ,t)=CR_{E}(λ)/R_{w}(λ)

Here, parameter C is an unknown amplitude, which depends inter alia upon the signal integration time and the distance of the instrument from the patient's ear drum. To eliminate the effect of parameter C, the normalized reflectivity spectrum is further normalized by a certain wavelength λ₀ from the incident light spectrum. In the present example, this is implemented by setting a relative spectrum: r(λ)=R(λ)/R(λ₀), so that r(λ₀)=1, wherein λ₀ is chosen in the center of the wavelength range of incident light (e.g., visible spectrum), and r(λ) is a “relative” spectrum, insofar as all intensities are measured relative to the intensity at λ₀.

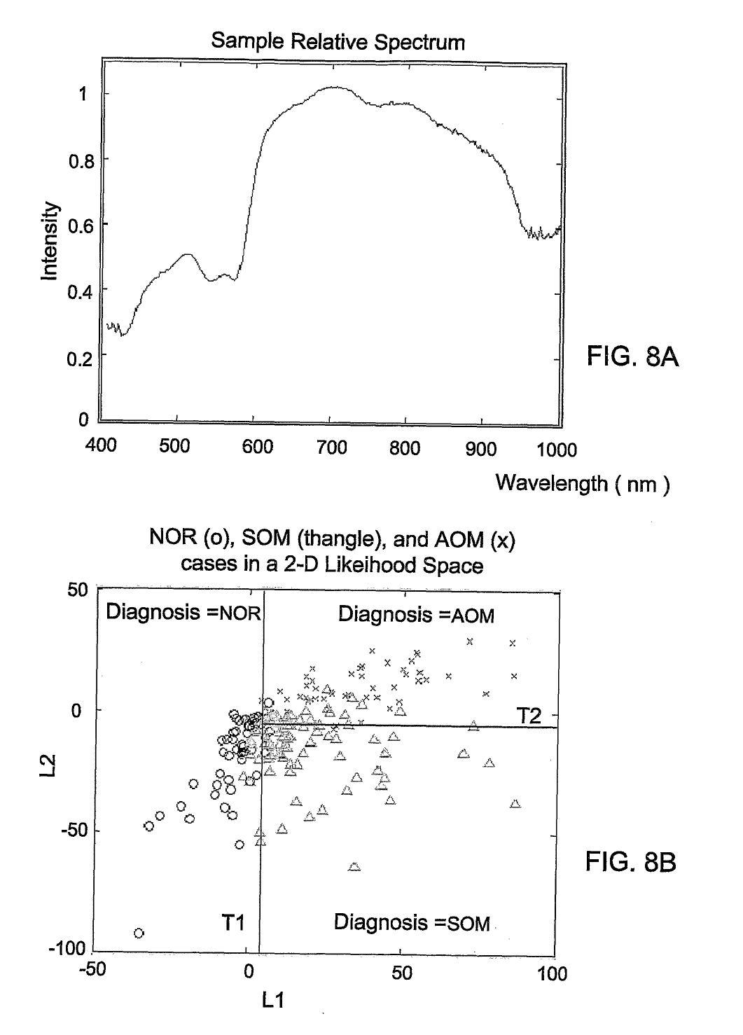

An example of the relative spectrum of a sample is shown in FIG. 8A. In this specific example of determining the patient's ear condition for the purposes of detecting the existence of serous otitis media (SOM) and acute otitis media (AOM), the normalized spectrum for 400-1000 nm is determined. The value of λ₀ is chosen in the center of this range, namely to be about 700 nm.

The inventors have found that a specific disease is characterized by at least one predetermined spectral range, from the entire measured spectrum, where the spectral behavior of the light response is maximally affected by the disease. For example, in order to detect the existence of serous otitis media (SOM) and acute otitis media (AOM), the spectral ranges of interest may be 500-650 nm (visible range) and 800-1 550 nm (IR range) respectively.

The processing of the relative spectrum r(λ) generally consists of applying the predetermined model to either the entire normalized spectrum or at least one selected region of this spectrum (region characterized by the maximal effects of a specific disease). In this specific example, the mathematical model utilizes a Likelihood Algorithm, and the processing consists of the following:

The relative spectrum r(λ) is sampled at certain discrete wavelengths, to generate a feature vector, r:
r={r(λₙ),n =1, 2 . . .N}

Two populations are considered: (A) healthy ears and (B) infected ears. By doing clinical tests on a large sample of ears of both types, the probability densities f (r|A) and f (r|B) are learned. For example, using Gaussian probability densities,
f(r|A)=g(r,μ_{A}, P_{A})
f(r|B)=g(r,μ_{B}, P_{B})
wherein

g(r,μ,P)=[2πdet(P)]^{-N/2}exp[−½(r−μ)^{T}P⁻(r−μ)]

μ=mean(r)

P=covariance(r)=N×N matrix

A new patient arrives, and his ear spectrum is measured. His feature vector is denoted by x. In order to diagnose the ear as healthy or infected, the algorithm forms the log-likelihood ratio:

$$\begin{matrix}L 1 ( \underline{x} ) = 2 log \{ f ( \underline{x} \vert B ) / f ( \underline{x} \vert A ) \} \\ = {( \underline{x} - {\underline{μ}}_{A} )}^{T} {P}_{A}^{- 1} ( \underline{x} - {\underline{μ}}_{A} ) - {( \underline{x} - {\underline{μ}}_{B} )}^{T} {P}_{B}^{- 1} ( \underline{x} - {\underline{μ}}_{B} )\end{matrix}$$

Then:

If L1(x)≦T1, diagnosis=A (healthy)

If L1(x)>T1, diagnosis=B (infected)

The numerical value of the threshold Ti is chosen to achieve a desired level of sensitivity (i.e., the probability of correctly diagnosing an infected ear).

Considering next the subdivision of infected ears into 2 classes: (B₁) serous otitis media (SOM) and (B₂) acute otitis media (AOM), if x is diagnosed as infected (L1(x)>T1), a second log-likelihood ratio is formed:

$$\begin{matrix}L 2 ( \underline{x} ) = 2 log \{ f ( \underline{x} \vert {B}_{2} ) / f ( \underline{x} \vert {B}_{1} ) \} \\ = {( \underline{x} - {\underline{μ}}_{B 1} )}^{T} {P}_{B 1}^{- 1} ( \underline{x} - {\underline{μ}}_{B 1} ) - {( \underline{x} - {\underline{μ}}_{B 2} )}^{T} {P}_{B 2}^{- 1} ( \underline{x} - {\underline{μ}}_{B 2} )\end{matrix}$$

and diagnosis is made as follows:

If L2(x)≦T2, diagnosis=B₁ (SOM)

If L2(x)>T2, diagnosis=B₂ (AOM)

wherein, again, T2 is a threshold which is chosen to achieve a desired level of sensitivity (i.e., the probability of correctly diagnosing AOM).

FIG. 8B shows the values of the measurable parameters L1 and L2 for normal (NOR), SOM and AOM conditions in the two-dimensional likelihood space, as obtained for the specific example of FIGS. 6A-6C.

The values L1 and L2 (log-likelihood) actually present the spectral factors being in the well-defined association with the value or range of values corresponding to the healthy condition of the ear and can thus be used by the physician for decision making. Preferably, these values L1 and L2 are further scaled to produce “spectral factors” that may be of better diagnostic value to a physician. They are continuous numbers that, over time and experience, may have value for borderline cases, much in the way that blood counts and iron levels in the blood are measured in continuous fashion. The following is the example of such scaling:
S1(x)=a1+b1*(L1(x)−T1)
S2(x)=a2+b2*(L2(x)−T2)

Here, coefficients a1, a2, b1 and b2 (that need not be necessarily different from each other) are appropriately selected to provide a scale which can be easily remembered by the physician, and thus facilitating the decision making.

The technique of the present invention thus provides for automatically determining the patient's condition by obtaining and analyzing the light response of the region of interest to predetermined wavelengths or wavelength ranges, and provides for effective collection of the light response. Output data presented to a physician (displayed on the monitor of the control unit) may include just the calculated spectral factor and preferably also the value or range of values for the spectral factor at normal (non-diseased) condition; or may include even clear “yes” and “no” results.

Those skilled in the art will readily appreciate that various modifications and changes can be applied to the embodiments of the invention as hereinbefore described without departing from its scope defined in and by the appended claims.

1. A measurement system for use in detecting a predetermined condition of a patient's ear indicative of a certain disease, the system comprising:
(a) an optical measuring unit configured and operable for irradiating a region of interest in the patient's ear with incident light including at least two different wavelengths, detecting light responses of the region of interest to said at least two different wavelengths, and generating measured data indicative thereof, said at least two different wavelengths being selected such that the light response of the region of interest to at least one first wavelength is substantially independent of said predetermined condition and the light response to at least one second wavelength is affected by said predetermined condition; and
(b) a control unit configured and operable for controlling operation of the optical measuring unit, and for receiving the measured data and processing it to generate output data indicative of whether or not said predetermined condition exists, the control unit comprising a memory utility for storing predetermined reference data indicative of the light response of the region of interest while in a healthy condition of the ear; a data processing and analyzing utility preprogrammed for processing and analyzing the measured data by determining a relation between the measured light responses and the corresponding reference data.

2. The system of claim 1, wherein said at least two wavelengths include the at least one first reference wavelength in at least one of the following wavelength ranges: about 700-900 nm and about 1420-1480 nm, and the at least one second operating wavelength in at least one of the following ranges: about 1200-1400 nm and 1500-1700 nm, the system being therefore operable for detecting a serous otitis media (SOM) condition of the patient's ear.

3. The system of claim 2, wherein said wavelengths include at least one additional second wavelength in at least one of the following wavelength ranges: about 540-550 nm and 570-580 nm, the system being therefore operable to detect an acute otitis media (AOM) condition.

4. The system of claim 2, wherein said wavelengths include at least one additional second wavelength in at least one of the following wavelength ranges: about 540-560 nm and 570-580 nm, the system being therefore operable to detect a change in a hemoglobin level in the region of interest.

5. The system of claim 1, wherein the reference data is indicative of a relation between the light responses of the healthy ear to said at least two different wavelengths.

6. The system of claim 5, wherein the measured data is in the form of a relation between the light responses of the region of interest in the patient's ear to said at least two different wavelengths.

7. The system of claim 2, wherein the reference data is indicative of the light response for the operating wavelength as a function of the light response for the reference wavelength corresponding to the healthy condition; the control unit being configured and operable to process the measured data to determine the light response for the operating wavelength as a function of the light response for the reference wavelength, I^{(W)}_{λoper}=f₁(I^{(W)}_{λref}), and determine a difference between the reference and measured data indicative of whether fluid media exists in the region of interest being therefore indicative of the SOM condition.

8. The system of claim 4, wherein the reference data is indicative of the light responses for the second operating wavelengths as functions of the light response for the reference wavelength corresponding to the healthy condition; the control unit being configured and operable to process the measured data to determine the light response for the second operating wavelength as a function of the light response for the reference wavelength, I^{(W)}_{λoper}=f₁(I_{λref}), and the light response for the additional second operating wavelength as a function of the light response for the reference wavelength, I^{(h)}_{λoper}=f₂(I_{λref}), and determine differences between the reference and measured data indicative of whether fluid media exists in the region of interest and whether there is a change in the hemoglobin concentration as compared to that of the healthy condition, being thereby indicative of the AOM condition.

9. The system of claim 1, wherein the reference data is indicative of a value or a range of values for at least one predetermined measurable parameter corresponding to a healthy condition of a patient.

10. The system of claim 1, wherein said measuring unit is configured and operable for spectrometric measurements, the measured data being therefore in the form of the light response of the region of interest as a function of wavelengths of the incident light.

11. The system of claim 1, wherein said measuring unit is configured and operable for spectrometric measurements, the measured data being therefore derived for said at least two different wavelengths from spectral data in the form of the light response of the region of interest as a function of wavelengths of the incident light.

12. The system of claim 9, wherein said measuring unit is configured and operable for spectrometric measurements, the measured data being therefore in the form of the light response of the region of interest as a function of wavelengths of the incident light.

13. The system of claim 1, wherein the measuring unit comprises an illumination unit configured and operable to generate said incident light, a light detection unit for detecting light of said at least two wavelengths and generating the measured data; and a light directing unit for directing the incident light to the region of interest and collecting light reflected from the region of interest, the light directing unit comprising an optical fiber arrangement.

14. The system of claim 13, wherein the light directing unit comprises a spectral filtering unit for separating said at least two wavelengths form the collected light.

15. The system of claim 13, wherein the fiber arrangement comprises at least one illuminating fiber for transmitting the incident light to the region of interest and at least one light collecting fiber for collecting and transmitting the reflected light to the detection unit.

16. The system of claim 15, wherein each of said at least two fibers has a predetermined numerical aperture such that for a given distance between the measuring unit and the region of interest said at least two wavelengths illuminate substantially the same spot in the region of interest and reflected light is collected substantially from the illuminated spot.

17. The system of claim 13, wherein the fiber arrangement is configured to provide substantially the same numerical aperture of light incidence onto the region of interest and light collection from the region of interest.

18. The system of claim 15, wherein the fiber arrangement is configured such that, lo for a given distance between the measuring unit and the region of interest, output of said at least one illuminating fiber presents a point-like light source for a predetermined spot size in the region of interest.

19. The system of claim 3, wherein the measuring unit comprises an illumination unit configured and operable to generate the incident light including the at least three different wavelengths, a light detection unit for detecting light of said at least three wavelengths and generating the measured data; and a light directing unit for directing the incident light to the region of interest and collecting light reflected from the region of interest, the light directing unit comprising an optical fiber arrangement.

20. The system of claim 19, wherein the light directing unit comprises and a spectral filtering unit.

21. The system of claim 19, wherein the illumination unit comprises a first light source generating light including the first reference wavelength in the range of about 1420-1480 nm and the second operating wavelength in the range of about 1500-1700, and a second light source generating light including the additional second wavelength in the range of 540-580 nm; and said detection unit comprises a first detector for detecting light of the first and second wavelength ranges, and a second detector for detecting light of the additional second wavelength range.

22. The system of claim 12, wherein the measuring unit is configured for determining a reference spectrum indicative of the light intensity illuminating the region of interest as a function of wavelengths of the incident light.

23. The system of claim 22, wherein the processing and analyzing utility is preprogrammed for processing the measured data by selecting a certain part of the measured data within at least one range of a predetermined light spectrum and applying a predetermined model to the selected part of the measured data to determine a corresponding value of said at least one predetermined measurable parameter for the measured patient and to generate output data indicative of association between the determined parameter value and the reference data.

24. The system of claim 23, wherein the processing of the measured spectral data comprises normalizing the measured spectral data by said reference spectrum, thereby obtaining a normalized reflectivity spectrum which then undergoes said processing by the predetermined model.

25. The system of claim 24, wherein the processing and analyzing of the measured data comprises optimizing the normalized reflectivity spectrum by further normalizing it by a certain wavelength λ₀ within said selected spectrum range, such that all the light intensities are measured relative to the intensity at wavelength λ₀, thereby obtaining a relative spectrum that undergoes said processing with the predetermined model.

26. The system of claim 23, wherein said at least one selected range of the predetermined light spectrum is defined by the patient's condition to be detected.

27. The system of claim 23, for use in determining the existence of otitis media condition in the patient's ear, the predetermined light spectrum being within a range of 300-1700 nm.

28. The system of claim 27, wherein the selected range of the predetermined light spectrum includes a range of 500-650 nm.

29. The system of claim 27, wherein the selected range of the predetermined light spectrum includes a range of 800-950 nm.

30. The system of claim 28, wherein the selected range of the predetermined light spectrum includes a range of 800-950 nm.

31. The system of claim 25, wherein said processing with the predetermined model comprises applying a Likelihood Algorithm to the relative spectrum, calculating a feature vector as a function of wavelengths within said selected range, and calculating a log-likelihood ratio between the feature vector of the relative spectrum and that of the reference data, said ratio presenting said at least one measurable parameters indicative of the patient's condition.

32. The system of claim 25, wherein said processing with the predetermined model lo comprises applying a Likelihood Algorithm to the relative spectrum, calculating a feature vector as a function of wavelengths within said selected range, and calculating a log-likelihood ratio between the feature vector of the relative spectrum and that of the reference data, said ratio being scalable to determine said at least measurable parameter indicative of the patient's condition.

33. The system of claim 23, wherein said control unit is configured as an expert system capable of timely analyzing the calculated measurable parameters and optimizing the model accordingly.

34. The system of claim 27, wherein the measuring unit is configured for determining a reference spectrum indicative of the light intensity illuminating the region of interest as a function of wavelengths of said predetermined incident light.

35. The system of claim 34, wherein the processing of the measured spectral data comprises normalizing the measured spectral data by said reference spectrum, thereby obtaining a normalized reflectivity spectrum which then undergoes said processing by the predetermined model.

36. The system of claim 35, wherein the processing and analyzing of the measured data comprises optimizing the normalized reflectivity spectrum by further normalizing it by a certain wavelength λ₀ within said selected spectrum range, such that all the light intensities are measured relative to the intensity at wavelength λ₀, thereby obtaining a relative spectrum that undergoes said processing with the predetermined model.

37. The system of claim 36, wherein said processing with the predetermined model comprises applying to a Likelihood Algorithm to the relative spectrum, calculating a feature vector as a function of wavelengths within said selected range, and calculating a log-likelihood ratio between the feature vector of the relative spectrum and that of the reference data.

38. The system of claim 36, wherein said processing with the predetermined model comprises applying a Likelihood Algorithm to the relative spectrum, calculating a feature vector as a function of wavelengths within said selected range, and calculating a log-likelihood ratio between the feature vector of the relative spectrum and that of the reference data, said ratio being scalable to determine said at least measurable parameter indicative of the patient's condition

39. The system of claim 37, wherein said processing of the relative spectrum comprises determining two measurable parameters indicative of the existence in the patient's ear of, respectively, serous otitis media (SOM) and acute otitis media (AOM).

40. The system of claim 35, wherein said normalizing of the measured spectral data by said reference spectrum comprises presenting the measured spectrum Eⱼ(λ,t) and the reference spectrum Wⱼ(λ,t) as, respectively,
Eⱼ(λ,t)=AIⱼ(λ,t)R_{E}(λ)Dⱼ(λ,t) and Wⱼ(λ,t)=BIⱼ(λ,t)R_{w}(λ)Dⱼ(λ,t)
wherein j is the number of the measuring unit, t is the time, λis the wavelength of incident light, A and B are unknown amplitudes, Iⱼ(λ,t) is the illumination spectrum of light source for the measuring unit j; Dⱼ(λ,t) is the light response spectrum of a detector assembly of for measuring unit j; R_{E}(λ) is the reflectivity spectrum of the region of interest; and R_{w}(λ) is the reflectivity of a reference surface used in obtaining said reference spectrum, the normalized reflectivity spectrum being thus determined as:
R(λ)=Eⱼ(λ,t)/Wⱼ(λ,t)=CR_{E}(λ)/R_{w}(λ),
wherein parameter C is a light signal amplitude depending inter alia upon a signal integration time and a distance between the measuring unit and the region of interest.

41. The system of claim 40, wherein the processing and analyzing of the measured relative spectrum comprises optimizing the normalized reflectivity spectrum by further normalizing it by a certain wavelength λ₀ within said selected spectrum range, such that all the light intensities are measured relative to the intensity at wavelength λ₀, thereby obtaining a relative spectrum in which the effect of parameter C is eliminated, said processing with the predetermined model being applied to the relative spectrum.

42. The system of claim 41, wherein said further normalizing comprises setting the relative spectrum r(λ)=R(λ)/R(λ₀) so that r(λ₀)=1.

43. The system of claim 41, wherein the selected value of λ₀ is the center of the wavelength range of said predetermined incident light.

44. The system of claim 43, wherein the creation of the reference data and the model comprises:
sampling a spectrum r(λ) at certain discrete wavelengths, to generate a feature vector r={ r(λₙ),n=1, 2 . . . N};
learning probability densities f (r|A) and f (r|B) for populations including (A) healthy ears and (B) infected ears; and
defining said value or range of values as a threshold T1 chosen to achieve a desired level of sensitivity corresponding to the probability of correctly diagnosing the existence of the predetermined condition of the patient's ear.

45. The system of claim 44, wherein the probability densities include Gaussian probability densities f (r|A)=g(r,μ_{A}, P_{A}) and f (r|B)=g(r,μ_{B}, P_{B}), wherein g(r,μ, P)=[2πdet(P)]^{-N/2}exp[−½(r−μ)^{T}P⁻¹ (r−μ)],μ=mean(r),P=covariance(r)=N×N matrix.

46. The system of claim 45, wherein the processing and analyzing of the relative spectrum comprises processing the measured feature vector to determine said at least one measurable parameters L1 as the log-likelihood ratio:
L1(x)=2log{f(x|B)/f(X|A)}=(x−μ_{A})^{T}P_{A}⁻¹(X−μ_{A})−(X−μ_{B})^{T}P_{B}⁻¹(X−μ_{B})

47. The system of claim 46, wherein the processing and analyzing of the relative spectrum comprises determining the association between said ratio and the predetermined threshold value T1 indicative of the existence of the otitis media in the patient's ear.

48. The system of claim 47, wherein said processing and analyzing provides for identifying wherein the otitis media includes serous otitis media (SOM) or acute otitis media (AOM).

49. The system of claim 48, wherein the creation of said reference data and said model comprises defining said value or range of values as a threshold T2 chosen to achieve a desired level of sensitivity corresponding to the probability of correctly diagnosing the existence of the serous otitis media (SOM) and acute otitis media (AOM).

50. The system of claim 49, wherein said processing and analyzing comprises:
processing the measured feature vector to determine another measurable parameter L2 as the log-likelihood ratio:
$$\begin{matrix}L 2 ( \underline{x} ) = 2 log \{ f ( \underline{x} \vert {B}_{2} ) / f ( \underline{x} \vert {B}_{1} ) \} \\ = {( \underline{x} - {\underline{μ}}_{B 1} )}^{T} {P}_{B 1}^{- 1} ( \underline{x} - {\underline{μ}}_{B 1} ) - {( \underline{x} - {\underline{μ}}_{B 2} )}^{T} {P}_{B 2}^{- 1} ( \underline{x} - {\underline{μ}}_{B 2} )\end{matrix}$$
determining the association between said ratio L2 and the predetermined threshold value T2 indicative of whether the detected otitis media is SOM or AOM.

51. The system of claim 1, wherein said measuring unit is configured as an optical probe for directing the at least two different wavelength to the region of interest along at least two separate channels, respectively, and directing the collected reflected light along at least one optical channel.

52. The system of claim 51, wherein the optical probe comprises a fiber bundle including at least two illuminating fibers defining said at least two channels, and at least one collecting fiber defining said at least one collecting channel.

53. The system of claim 52, wherein at least within a distal end of the probe, by which it is to be brought to the region of interest, the at least one collecting fiber is located within a region between the at least two illuminating fibers.

54. The system of claim 53, wherein the collecting fiber has a cross section larger than the illuminating fiber.

55. The system of claim 52, wherein the probe is configured to define an imaging channel.

56. The system of claim 55, wherein at least within a distal end of the probe, by which it is to be brought to the region of interest, the fiber bundle is located in central region of the probe; said imaging channel being defined by at least one of the at least two illuminating fibers that transmits the light to the region of interest and a periphery region of the probe surrounding the fiber bundle for transmitting the reflected from the region of interest.

57. The system of claim 1, wherein said measuring unit is configured as an optical probe for transmitting light emanating from a target tissue in the region of interest along at least two separate optical channels.

58. The system of claim 57, wherein the probe comprises a probe head and a speculum member removably fitted to a distal end of said probe head, wherein said probe head comprises light transmission unit for directing the incident light to said target tissue via a distal end of said speculum, and for directing light emanating from said target tissue along said at least two separate optical channels; and wherein said speculum member is adapted for positioning said distal end thereof proximate to the target tissue.

59. The system of claim 58, wherein said distal end of said speculum member comprises an optical aperture for enabling illuminating light and emanating light to pass therethrough from and to said optical probe.

60. The system of claim 59, wherein said at least two separate optical channels comprise:
a first channel for enabling qualitative analysis of the collected light emanating from said target tissue; and
a second channel for enabling quantitative analysis of the collected light emanating from said target tissue.

61. The system of claim 60, wherein said speculum member comprises an internal reflecting mirror for directing illuminating light from said light transmission means to said distal end.

62. The system of claim 57, wherein said probe head comprises a beam splitter arrangement for splitting light traveling in a proximal direction from said distal end into said first channel and said second channel.

63. The system of claim 62, wherein said beam splitter arrangement comprises a parabolic mirror having an aperture therein.

64. The system of claim 63, wherein said aperture is configured for directing a first portion of said light traveling from said distal end therethrough along said first channel and towards an objective.

65. The system of claim 64, wherein said objective comprises an eyepiece ocular.

66. The probe of claim 64, wherein said objective comprises a suitable camera means for recording said image.

67. The system of claim 63, wherein said parabolic mirror comprises an optical focusing element for directing a second portion of said light traveling from said distal end along said second channel and towards a light sensor.

68. The system of claim 59, wherein said speculum member comprises a suitable first waveguide for directing illuminating light from said light transmission means to said distal end.

69. The system of claim 68, wherein said first waveguide is in the form of a first layer of material having waveguiding properties comprised in said speculum member, said first layer having a transmitting face proximate to said distal end, and a first mating face in optical communication with said transmitting face and adapted for enabling illumination light from said light transmission means to pass therethrough to said transmitting face when said speculum member is fitted to said probe head.

70. The system of claim 69, wherein said light transmission means comprises a second mating face configured to provide optical communication between said light transmission means and said first mating face when said speculum member is fitted to said probe head.

71. The system of claim 69, wherein said first channel is in the form of a proximal aperture comprised in said probe head, said aperture being configured for directing a first portion of said light traveling from said distal end therethrough and towards an objective.

72. The system of claim 70, wherein said first channel is in the form of a proximal aperture comprised in said probe head, said aperture being configured for directing a first portion of said light traveling from said distal end therethrough and towards an objective.

73. The system of claim 72, wherein said objective comprises an eyepiece ocular.

74. The system of claim 72, wherein said objective comprises a suitable camera means for recording said image.

75. The system of claims 69, wherein said first layer is made from PMMA.

76. The system of claims 60, wherein said second channel comprises a suitable second waveguide for directing light from said distal end towards a light sensor.

77. The system of claims 68, wherein said second waveguide is in the form of a second layer of material having waveguiding properties comprised in said probe head, said second layer having a second receiving face proximate to said distal end, and a second transmitting face in optical communication with said second receiving face and adapted for enabling light from outside of said distal end to pass therethrough from said second receiving face to said second transmitting face.

78. The system of claim 77, wherein said second transmitting face is in optical communication with a suitable light sensor.

79. The system of claim 77, wherein said second layer is made from PMMA.

80. The system of claim 78, wherein said second layer is made from PMMA.

81. The system of claim 77, wherein said light sensor is operatively connected to a suitable spectrometer.

82. The system of claim 79, wherein said light sensor is operatively connected to a suitable spectrometer.

83. The system of claim 80, wherein said light sensor is operatively connected to a suitable spectrometer.

84. The system of claim 57, wherein said speculum member is disposable after use with one patient.

85. The system of claim 57, wherein said speculum member is adapted for positioning said distal end thereof within an ear canal of a patient proximate to the ear drum thereof.

86. The system of claim 57, wherein said speculum member is adapted for positioning said distal end thereof within a vaginal canal of a patient proximate to the ear drum thereof.

87. The system of claim 57, wherein said speculum member further comprises a plug removably fitted to said distal aperture, said plug configured to diffusedly reflect incident light thereon from said light transmission means in a known manner.

88. A measurement system for use in determining a patient's condition, the system comprising:
(a) an optical measuring unit operable for applying spectral measurements to the region of interest in a patient's body with predetermined light spectrum and producing measured spectral data indicative thereof; and
(b) a control unit for receiving and processing the measured data to generate output data indicative of the measurement results, the control unit comprising a memory utility for storing predetermined reference data representative of a value or a range of values for at least one predetermined measurable parameter corresponding to a healthy condition of a patient; a data processing and analyzing utility preprogrammed for processing and analyzing the measured data by selecting a certain part of the measured data within at least one range of the predetermined light spectrum and applying a predetermined model to the selected part of the measured data to determine a corresponding value of said at least one predetermined measurable parameter for the measured patient and to generate said output data indicative of association between the determined parameter value and the reference data.

89. An optical probe for transmitting light emanating from a target tissue in a region of interest along at least two separate optical channels, comprising a probe head and a speculum member removably fitted to a distal end of said probe head, wherein:
said probe head comprises light transmission means for directing an illuminating light to said target tissue via a distal end of said speculum, and means for directing light emanating from said target tissue along at least two separate optical channels; and wherein
said speculum member is adapted for positioning said distal end thereof proximate to the target tissue.

90. A control unit configured for receiving spectral measured data from a region of interest on a patient's body, and processing the received data to generate output data indicative of the patient's condition, the control unit comprising a memory utility for storing predetermined reference data representative of a value or a range of values for at least one predetermined measurable parameter corresponding to a healthy condition of a patient and for storing the reference spectrum; a data processing and analyzing utility preprogrammed for processing and analyzing the measured data by:
obtaining a relative spectral, said obtaining including selecting a part of the measured data within at least one range of the predetermined light spectrum and normalizing said selected part of the measured data by the reference spectrum;
applying a predetermined model to the relative spectrum to determine a corresponding value of said at least one predetermined measurable parameter for the measured patient and generate said output data indicative of association between the determined parameter value and the reference data.

91. A measurement system for use in determining the patient's condition, the system comprising an optical measuring unit operable for carrying out spectral measurements, the measuring unit comprising a light source system for generating light of predetermined wavelengths, a detector for collecting light impinging thereon and generating data indicative thereof, said measuring unit comprising a plug that is shiftable between its operative and inoperative positions so as to be, respectively, in and out of the optical path of light propagating from the light source system and having a highly diffusedly reflective surface, the measuring unit being selectively operable to apply spectral measurements to said surface and obtain reference spectrum data indicative of the reflectance of incident light from said surface and to apply spectral measurements to the region of interest on patient's body to obtain measured spectral data indicative of the reflectance of the incident light from the region of interest.

92. The system of claim 91, comprising a control unit for receiving and processing the measured data to generate output data indicative of the measurement results, the control unit comprising a memory utility for storing predetermined reference data representative of a value or a range of values for at least one predetermined measurable parameter corresponding to a healthy condition of a patient and for storing the reference spectrum; a data processing and analyzing utility preprogrammed for processing and analyzing the measured data by
obtaining a relative spectral, said obtaining including selecting a part of the measured data within at least one range of the predetermined light spectrum and normalizing said selected part of the measured data by the reference spectrum;
applying a predetermined model to the relative spectrum to determine a corresponding value of said at least one predetermined measurable parameter for the measured patient and generate said output data indicative of association between the determined parameter value and the reference data.

93. A program storage device readable by machine, tangibly embodying a program of instructions executable by the machine to perform method steps for receiving spectral data measured from a region of interest on a patient's body, and processing the received data to generate output data indicative of the patient's condition, the storage device comprising a memory utility for storing predetermined reference data representative of a value or a range of values for at least one predetermined measurable parameter corresponding to a healthy condition of a patient and for storing the reference spectrum; a data processing and analyzing utility preprogrammed for processing and analyzing the measured data by obtaining a relative spectral, said obtaining including selecting a part of the measured data within at least one range of the predetermined light spectrum and normalizing said selected part of the measured data by the reference spectrum; applying a predetermined model to the relative spectrum to determine a corresponding value of said at least one predetermined measurable parameter for the measured patient and generate said output data indicative of association between the determined parameter value and the reference data.

94. A computer program product comprising a computer useable medium having computer readable program code embodied therein for processing spectral data measured from a region of interest on a patient's body, the computer program product comprising: a data processing and analyzing utility for selecting a part of the measured data within at least one predetermined range of a light spectrum used in the measured data and utilizing a reference spectrum to normalize said selected part of the measured data to obtain a relative spectrum, applying a predetermined model to the relative spectrum and utilizing reference data representative of a value or a range of values for at least one predetermined measurable parameter corresponding to a healthy condition of a patient, to thereby determine a value of said at least one predetermined measurable parameter corresponding to the measured data and generate output data indicative of association between the determined parameter value and the reference data.

95. A method for processing spectral measured data to enable determination of a patient's condition, the method comprising processing the spectral measured data indicative of reflection of predetermined incident light from a region of interest as a function of wavelengths of the incident light; said processing comprising selecting a predetermined part of the measured spectral data corresponding to at least one range of the predetermined incident light, normalizing the selected measured data to obtain a relative spectrum, and applying a predetermined model to the relative spectrum to determine a corresponding value of at least one predetermined measurable parameter and to generate output data indicative of association between the determined parameter value and preset reference data, said reference data being representative of a value or a range of values for said at least one predetermined measurable parameter corresponding to a healthy condition of a patient.

96. A method for use in detecting an SOM or AOM condition of a patient's ear, the method comprising illuminating a region of interest in the ear by at least two wavelengths, detecting light responses of the region of interest to said at least two different wavelengths, and generating measured data indicative thereof, said at least two different wavelengths being selected such that the light response of the region of interest to at least one first wavelength is substantially independent of said condition and the light response to at least one second wavelength is affected by said condition.

97. A method for use in detecting an SOM or AOM condition of a patient's ear, the method comprising illuminating a region of interest in the middle ear by at least two wavelengths, detecting light responses of the region of interest to said at least two different wavelengths, and generating measured data indicative thereof, said at least two different wavelengths being selected such that at least one first wavelength satisfies at least one of the following: is substantially absorbable by water or is substantially transmittable by water, the light response to said first wavelength being therefore substantially independent of said condition, and the at least one second wavelength being partially absorbable by water the light response to said at least one second wavelength being therefore affected by said condition.

98. A method for use in detecting an SOM or AOM condition of a patient's ear, the method comprising illuminating a region of interest in the middle ear by at least two wavelengths, detecting light responses of the region of interest to said at least two different wavelengths, and generating measured data indicative thereof, said at least two different wavelengths being selected such that at least one first wavelength satisfies at least one of the following: is substantially absorbable by water or is substantially transmittable by water, the light response to said first wavelength being therefore substantially independent of said condition, and the at least one second wavelength being partially absorbable by water the light response to said at least one second wavelength being therefore affected by said condition, a change in the intensity of the detected light of said at least one second wavelength from a corresponding intensity for a healthy condition being indicative of the SOM or AOM condition, and a decrease in the intensity of the detected light to said at least one second wavelength from that corresponding to the SOM condition being indicative of the AOM condition.

99. A method for use in detecting an SOM or AOM condition of a patient's ear, the method comprising illuminating a region of interest in the middle ear by at least three wavelengths, detecting light responses of the region of interest to said at least three different wavelengths, and generating measured data indicative thereof, said at least three different wavelengths being selected such that at least one first wavelength satisfies at least one of the following: is substantially absorbable by water and substantially non-absorbable by hemoglobin, and is substantially transmittable by water and substantially non-absorbable by hemoglobin; the light response of the region of interest to said at least one first wavelength being therefore substantially independent of said condition, the at least two second wavelengths including a wavelength that is partially absorbable by water and a wavelength that is relatively highly absorbable by hemoglobin the light response to said at least two second wavelengths being therefore affected by said condition.

100. A method for use in determining a patient's condition, the method comprising:
(i) providing reference data representative of a value or a range of values for at least one predetermined measurable parameter corresponding to a healthy condition of a patient, and a certain reference spectrum corresponding to reflectance of a predetermined light spectrum from a reference highly reflective surface;
(ii) applying spectral measurements to a region of interest on the patient's body with predetermined light spectrum and producing measured spectral data indicative thereof; and
(iii) processing the measured data to generate output data indicative of the measurement results, said processing comprising selecting a part of the measured data within at least one range of the predetermined light spectrum and applying a predetermined model to the selected part of the measured data to determine a corresponding value of said at least one predetermined measurable parameter for the measured patient and generate said output data indicative of association between the determined parameter value and the reference data.
